(19) United States
(12) Patent Application Publication
(10) Pub. No.: US 2010/0143250 A1
(43) Pub. Date: Jun. 10, 2010

(54) COMPOUNDS FOR THE DIAGNOSIS OF APOPTOSIS

(76) Inventors: Marc Port, Deuil La Barre (FR); Olivier Rousseaux, Senlis (FR); Robert Muller, Mons (BE); Carmen Burtea, Mons (BE)

Correspondence Address: BIRCH STEWART KOLASCH & BIRCH, PO BOX 747, FALLS CHURCH, VA, 22040-0747, US

(21) Appl. No.: 12/593,495

(22) PCT Filed: Mar. 21, 2008
(86) PCT No.: PCT/EP2008/053447

§ 371 Date: Sep. 28, 2009

(30) Foreign Application Priority Data
Mar. 28, 2007 (FR) 0754086

(51) Int. Cl. A61K 51/08; C07K 7/06; A61K 49/14; A61K 49/00

(52) U.S. Cl. 424/1.69; 530/329; 424/9.323; 424/9.341; 424/9.1; 977/773; 977/927; 977/930; 977/774

(57) ABSTRACT

The present invention relates to a compound of general formula (I) below: Signal−Linker−Peptide (I) in which Signal represents a signal entity; Linker, which may or may not be present, represents a chemical bond and Peptide represents a peptide comprising an apoptosis-targeting peptide, the apoptosis-targeting peptide being chosen from the peptides having the formula below and the functional equivalents thereof:
X1-X2-X3-X4-X5-X6 (1) (SEQ ID No 1) in which X1 and X2 represent, independently of one another, leucine or isoleucine, X3 and X4 represent lysine, X5 represents proline and X6 represents phenylalanine, advantageously the peptide L-I-K-K-P-F (SEQ ID No 11) and the functional equivalents thereof;
D-A-H-S-X7-S (2) (SEQ ID No 2) in which X7 represents phenylalanine or leucine;
P-G-D-L-X8-X9 (3) (SEQ ID No 3) in which X8 represents serine or valine and

X9 represents threonine or arginine;
H-G-X10-L-S-X11 (4) (SEQ ID No 4) in which X10 represents aspartic acid or histidine, and X11 represents threonine or isoleucine;
V-L-G-E-R-G (5) (SEQ ID No 5);
   and the pharmaceutically acceptable salts of these compounds.

The invention relates to novel compounds for diagnosing diseases related to apoptosis, to the method for the preparation thereof and to the use thereof in medical imaging.

Apoptosis, or programmed cell death, refers to the mechanisms which result in the suicide of eukaryotic cells in response to certain stimuli. This physiological phenomenon provides the balance between cells that proliferate and those that should be naturally eliminated. In a pathological situation, it may be increased and have devastating consequences in functional terms in certain tissues. The role of apoptosis has been widely demonstrated in chronic neurodegenerative diseases (Alzheimer's disease, Parkinson's disease), in ischemic diseases (myocardial infarction, cerebral ischemia) and in inflammatory pathological conditions (atherosclerosis, arthritis). Conversely, apoptosis may play a beneficial role when it results in the death of tumor cells during treatments by radiotherapy or by chemotherapy.

The detection of apoptosis by imaging constitutes a novel diagnostic tool that is very useful for helping clinicians to assess the severity of a pathological condition, to predict the risk of occurrence of a serious event and to monitor the effectiveness of treatments through a fine characterization of the lesions.

The detection of apoptosis in vivo is difficult owing in particular to the fact that the phenomenon is not always expressed, and that it is relatively brief since it occurs over a period of a maximum of 6 to 24 hours, after which time the cell debris is eliminated by the phagocytic cells.

The prior art describes several attempts to develop contrast agents intended to target apoptotic cells. These agents typically comprise a targeting portion known as biovector, intended for the specific recognition of the biological target associated with a mechanism of apoptosis, and a signal portion capable of being detected by imaging methods: optical imaging, scintigraphy, magnetic resonance imaging (MRI). The signal portion is, for example, a linear or macrocyclic chelate of a paramagnetic metal such as gadolinium, in particular DTPA, DTPA BMA, DTPA BOPTA, DO3A or DOTA.

As regards the targeting portion, the particularity of apoptotic cells of exposing phosphatidylserine (PS) on the outer leaflet of the plasma membrane (whereas it is normally confined to the inner leaflet) has led to biovectors that target PS being sought. The externalization of PS occurs early and may therefore play the role of a marker for the apoptotic process.

Several proteins have a high affinity and a high specificity for PS. This is in particular the case with annexin V. It is an endogenous protein with a molecular weight of 35 kDa, present at the intracellular or extracellular level. It has been coupled to various reporter groups depending on the imaging methods selected. In particular, it has been conjugated to superparamagnetic particles of iron oxide (Schellenberger E A. et al., Mol. Imaging, 2002, 1:102-107). In vitro, this contrast agent has made it possible to obtain an MRI image in Jurkat cells made apoptotic after treatment with camptothecin.

Currently, the most commonly used method for detecting apoptosis in vivo is scintigraphy using annexin V coupled to 99mTc. In a model of atherosclerosis in rabbit subjected to deendothelialization of the abdominal aorta and to a cholesterol-rich diet, the marker accumulated massively in the lesions (Kolodgie F D. et al., Circulation, 2003, 108:3134-3139).

Attempts are continually being made to improve the quality of in vivo diagnosis using novel, very specific markers intended for the imaging methods known to those skilled in the art, in particular MRI, X-rays, gamma-ray scintigraphy, CT scan, ultrasound, PET and optical imaging. It is recalled that, in the case of MRI, a contrast is obtained through the administration of contrast agents containing paramagnetic metals or superparamagnetic compounds which have an effect on the relaxivity of the protons of water. In the case of scintigraphy, the contrast is obtained by the specific localization of a radiopharmaceutical compound emitting gamma- or beta-rays.

Compounds for which the chemical synthesis is not too complex, which are sufficient stable in vivo for use in medical imaging, and the cost price of which is not too high, and therefore in particular compounds which do not comprise a protein biovector such as annexin, are sought, in particular for MRI.

Products which allow imaging of the early signs of apoptosis, in order in particular to aid with selecting the most effective chemotherapy treatments, are also sought for oncology. The activity of antihormonal drugs and anti-angiogenic drugs and radiotherapy depend mainly on apoptosis of the tumor cells, and for this reason, the monitoring of this phenomenon by imaging will provide essential information for the treatment of patients.

In cerebral or cardiac ischemic diseases, apoptosis generally occurs 24 to 48 hours after the initial attack. Anti-apoptotic medicaments, acting, for example, on the caspase pathway, would certainly be useful if they could be combined with an imaging method. The same is true with regard to the use of immunosuppressive drugs in the case of organ transplant rejection. There are therefore many indications for a contrast agent capable of effectively detecting apoptotic cells in vivo.

The applicant has succeeded in obtaining compounds comprising a portion for targeting regions of apoptosis, which are effective not only in vitro, but also and especially in MRI in vivo. Such compounds are in fact difficult to obtain since it is necessary not only to identify a biovector that is effective in vitro, but also to obtain a compound that is effective in human clinical diagnostic imaging. This is particularly the case for MRI, which is recognized as being a highly sought-after technique since it does not use radioactivity, but the sensitivity of which is very much lower than that of nuclear medicine.

The compound obtained should at the same time have sufficient affinity to recognize its target, a high specificity so as to be a distinctive indicator of the pathological state and an appropriate stability so as not to be degraded or modified in vivo, and in addition without the signal portion interfering so as to impair these various parameters (for example, without impairing the affinity and the stability of the ligand).

In the present application, the following correspondance table is used.

| | | |
|---|---|---|
| Alanine | A | Ala |
| Arginine | R | Arg |
| Asparagine | N | Asn |
| Aspartate | D | Asp |
| Cysteine | C | Cys |
| Glutamate | E | Glu |
| Glutamine | Q | Gln |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

After many attempts, the applicant has succeeded in obtaining compounds that are effective in vivo.

The invention thus relates to a compound of general formula (I) below:

Signal-Linker-Peptide (I)

in which:
Signal represents a signal entity;
Linker, which may or may not be present, represents a chemical bond, and
Peptide represents a peptide comprising an apoptosis-targeting peptide, the apoptosis-targeting peptide being chosen from the peptides of formula below and the functional equivalents thereof:

| | |
|---|---|
| X1-X2-X3-X4-X5-X6 (1) | (SEQ ID No 1) |

in which X1 and X2 represent, independently of one another, leucine or isoleucine, X3 and X4 represent lysine, X5 represents proline and X6 represents phenylalanine, advantageously the peptide L-I-K-K-P-F (SEQ ID No 11) and functional equivalents thereof;

| | |
|---|---|
| D-A-H-S-X7-S (2) | (SEQ ID No 2) |

in which X7 represents phenylalanine or leucine;

| | |
|---|---|
| P-G-D-L-X8-X9 (3) | (SEQ ID No 3) |

in which X8 represents serine or valine and X9 represents threonine or arginine;

| | |
|---|---|
| H-G-X10-L-S-X11 (4) | (SEQ ID No 4) |

in which X10 represents aspartic acid or histidine, and X11 represents threonine or isoleucine;

| | |
|---|---|
| V-L-G-E-R-G (5); | (SEQ ID No 5) |

and the pharmaceutically acceptable salts of these compounds.

The applicant has in fact demonstrated the affinity of the peptides of formula (1) to (5) for PS as will be described below.

The expressions “apoptosis-targeting peptide” and “disease associated with apoptosis” are intended to mean that the applicant's compounds are compounds capable of targeting biological regions (cells, tissues, organs, etc.) undergoing a mechanism of apoptosis, this mechanism being reflected by a disease associated with apoptosis at an early or already advanced stage. This diagnostic information then allows the clinician to provide a more suitable treatment, such as the administration of an anti-apoptotic medicament suitable for the biological area concerned, and, where appropriate, possible supplementary diagnostics. Thus, the invention relates to compounds of formula I and to the use thereof for targeting (one or more) biological region(s) (cells, tissues, organs, etc.) undergoing a mechanism of apoptosis.

The expression “apoptosis-targeting peptide” is also denoted PEPTIDE P in the application. Advantageously, Peptide represents a PEPTIDE P.

The expression “LIKKPF (SEQ ID No 11) and the defined functional equivalents thereof” is intended to mean the peptide LIKKPF (SEQ ID No 11), the effectiveness of which has been demonstrated by the applicant, and the derived peptides of formula (1) X1-X2-X3-X4-X5-X6 (SEQ ID No 1) which exhibit an effectiveness in imaging which is similar to or better than LIKKPF (SEQ ID No 11) (which includes the peptidomimetics), this effectiveness being tested by means of tests and models in vivo, described in detail in the application, or of suitable analogous models. Among these peptides, mention will be made of the following derived peptides X1-X2-X3-X4-X5-X6 (SEQ ID No 6), with the proviso that they exhibit an effectiveness in imaging that is similar to or better than LIKKPF (SEQ ID No 11):
X1 is leucine L or another alkyl-substituted hydrophobic amino acid (isoleucine, alanine, valine),
X2 is isoleucine I or another allcyl-substituted hydrophobic amino acid (leucine, alanine, valine),
X3 and X4 are lysine K or another amino acid comprising a basic function (arginine, histidine, ornithine), but preferably lysine, which is very advantageous for the interaction of the peptide with its target,
X5 is proline P,
X6 is phenylalanine F or another hydrophobic aromatic amino acid (for example, tryptophan, tyrosine, histidine, biphenylalanine, naphthylalanine).

Likewise for the other peptides of formulae (2) to (5), the functional equivalents are analogous or derived peptides which exhibit an effectiveness in imaging that is similar to or better than that of the peptide under consideration of formula (2) to (5).

The invention also relates to the compounds of formula (I) functionally equivalent to the compound of formula (I), i.e. having an vitro and in vivo activity comparable to these compounds, the affinity and the specificity of the peptides equivalent to the Peptide of the compound (I) being evaluated by appropriate screening methods, such as that described in the detailed description.

The expression “peptide comprising at least one apoptosis-targeting peptide (PEPTIDE P)” is intended to mean a peptide having the peptide sequence for recognition of the biological target of the PEPTIDE P, optionally flanked at the N- and/or C-terminal end by a chemical group which does not interfere with this sequence.

In the present application, the apoptosis-targeting peptide and derivatives thereof are denoted “PEPTIDE P”.

Advantageously, the following peptides are excluded from the peptides P according to the invention:

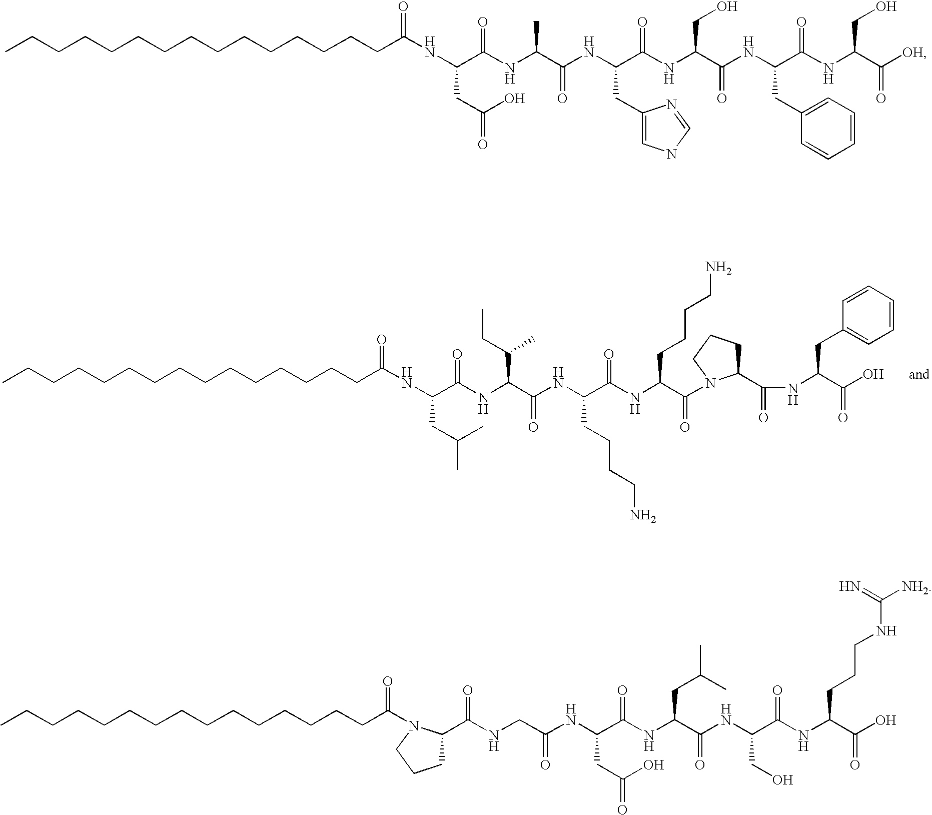

These peptides are in particular excluded when Signal represents a lipid nanoparticle.

The term “Signal entity” is intended to mean a chemical entity which makes it possible to obtain a signal in medical imaging, in particular:
a chelate capable of being coupled to a paramagnetic metal or to a radionucleide,
a metal nanoparticle, in particular a superparamagnetic nanoparticle of iron oxide,
a lipid nanoparticle, advantageously in the form of an emulsion, this nanoparticle carrying at least one chelate capable of being coupled to a paramagnetic metal (in this case, the PEPTIDE P is grafted to the lipid nanoparticle in emulsion, which itself carries chelates; the bonding of the peptide to the lipid nanoparticle is, for example, carried out by means of a chemical linking group).

In one advantageous embodiment, Signal represents a signal entity chosen from:
a chelate which may or may not be coupled to a paramagnetic metal M or to a radionucleide,
a superparamagnetic nanoparticle coated with an organic layer, preferably having an iron oxide core, and
a label for optical imaging.

Advantageously, the following compounds are excluded from the compounds of formula I according to the present invention:

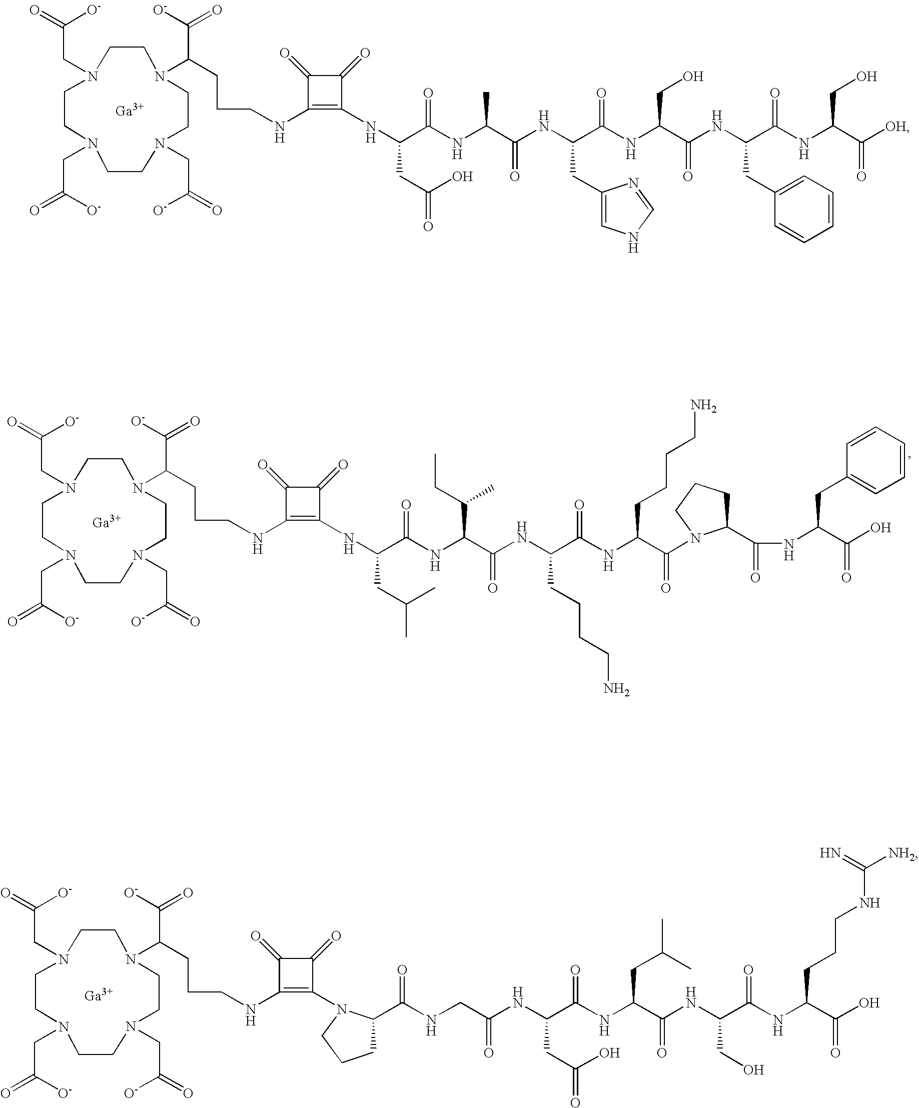

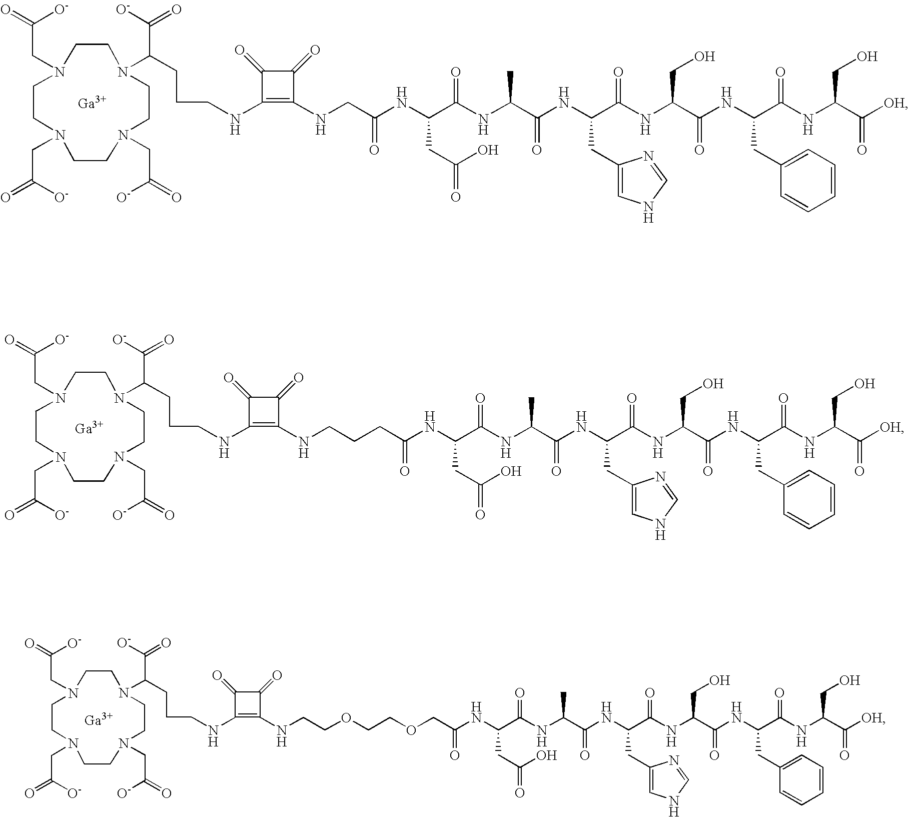

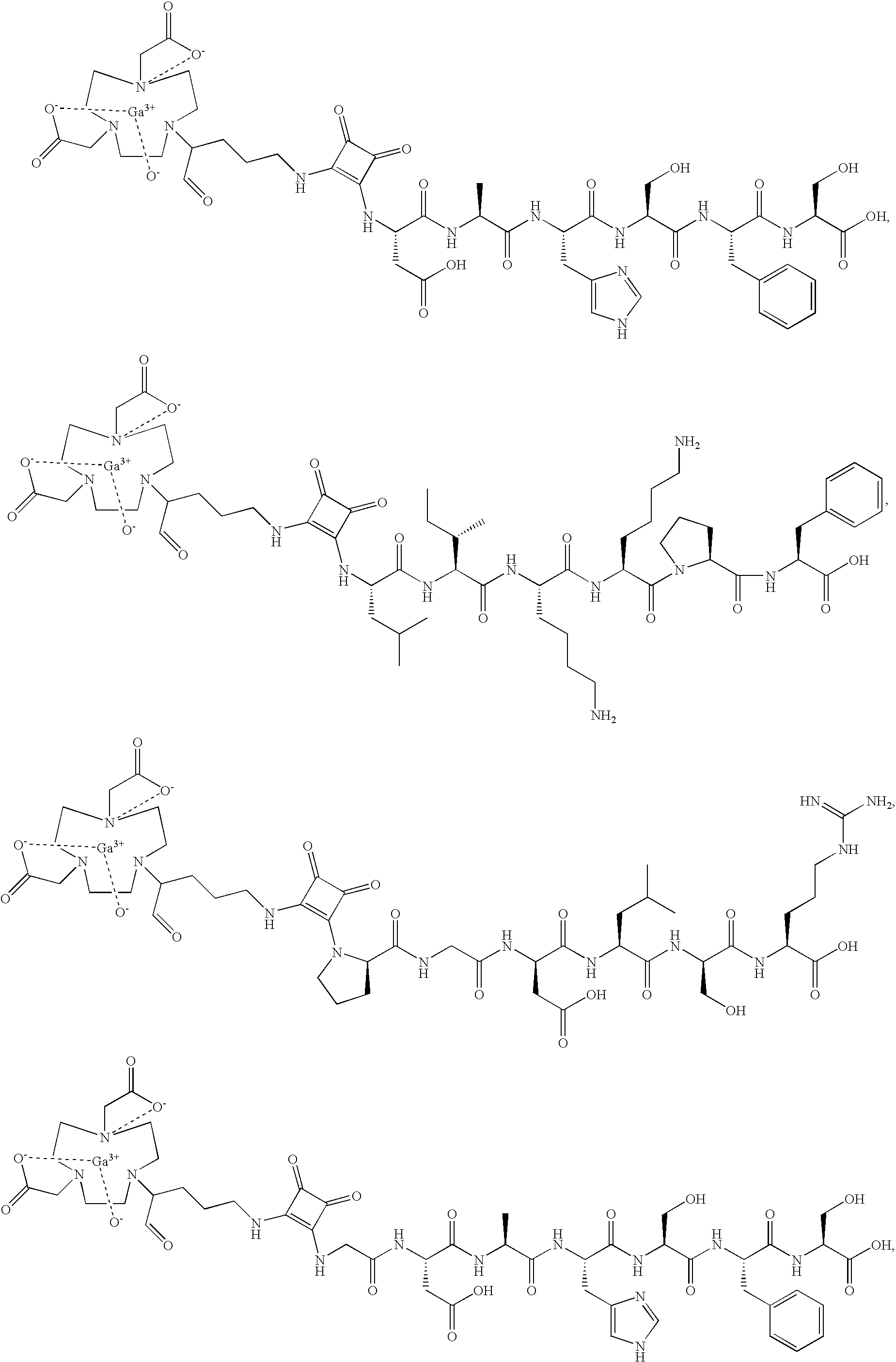

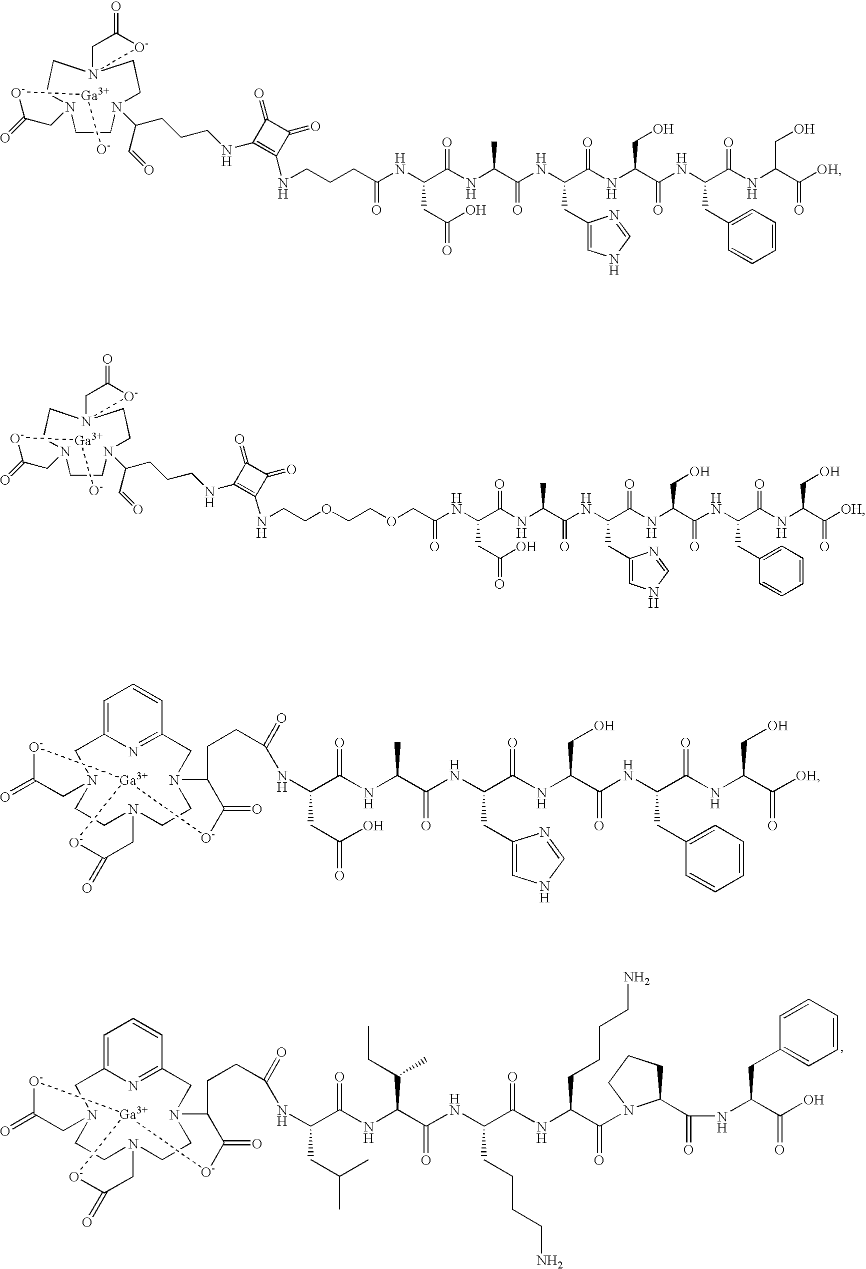

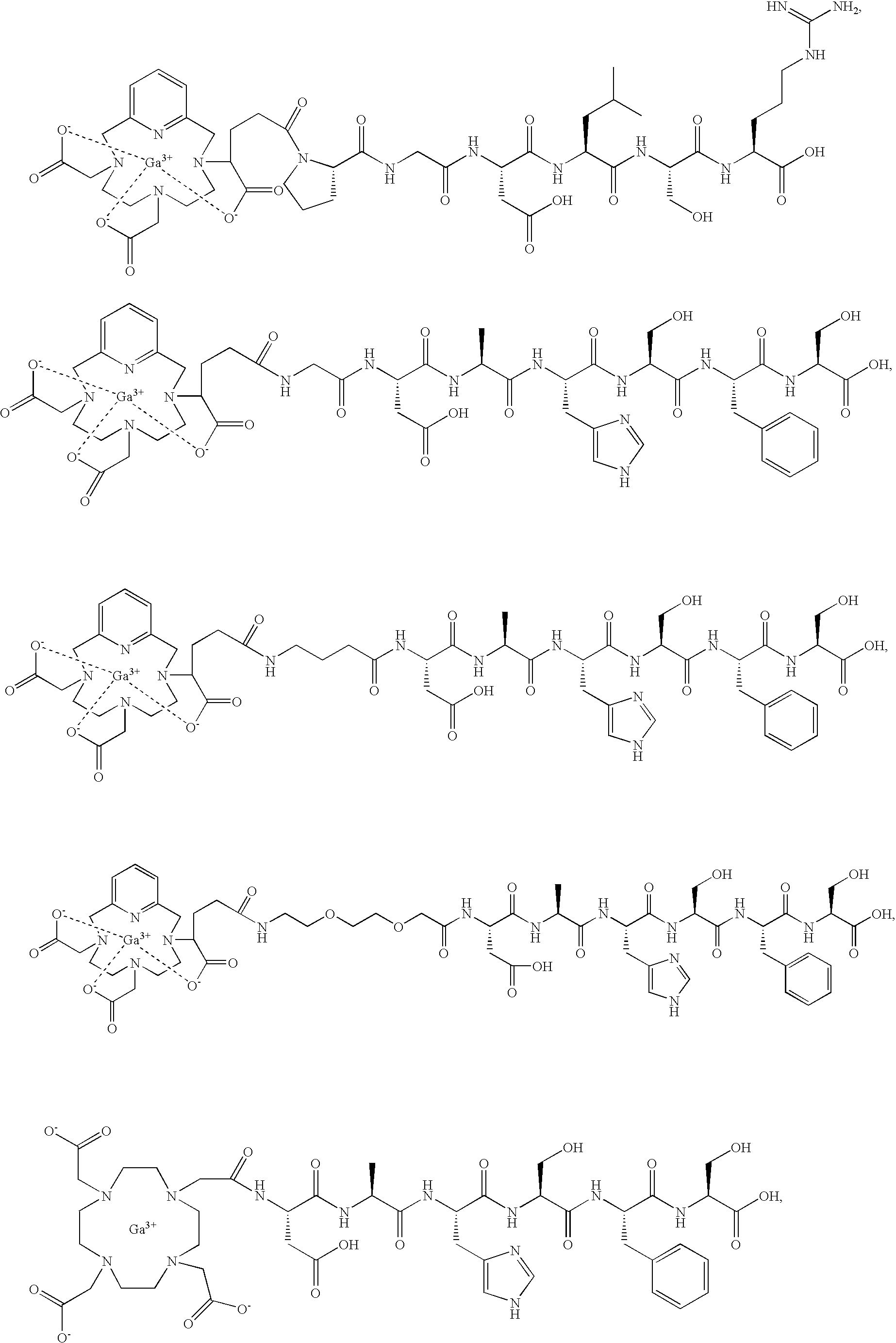

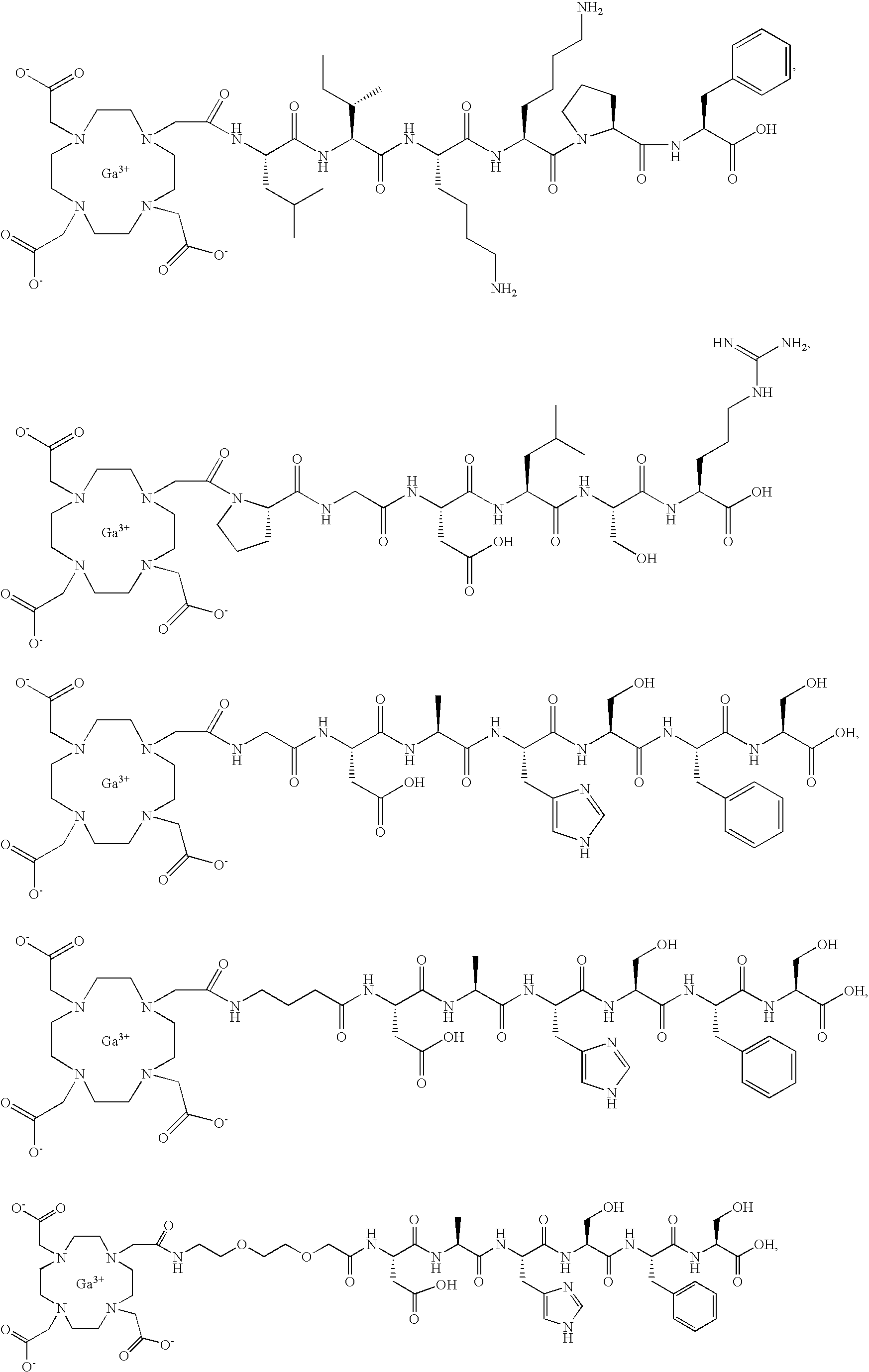

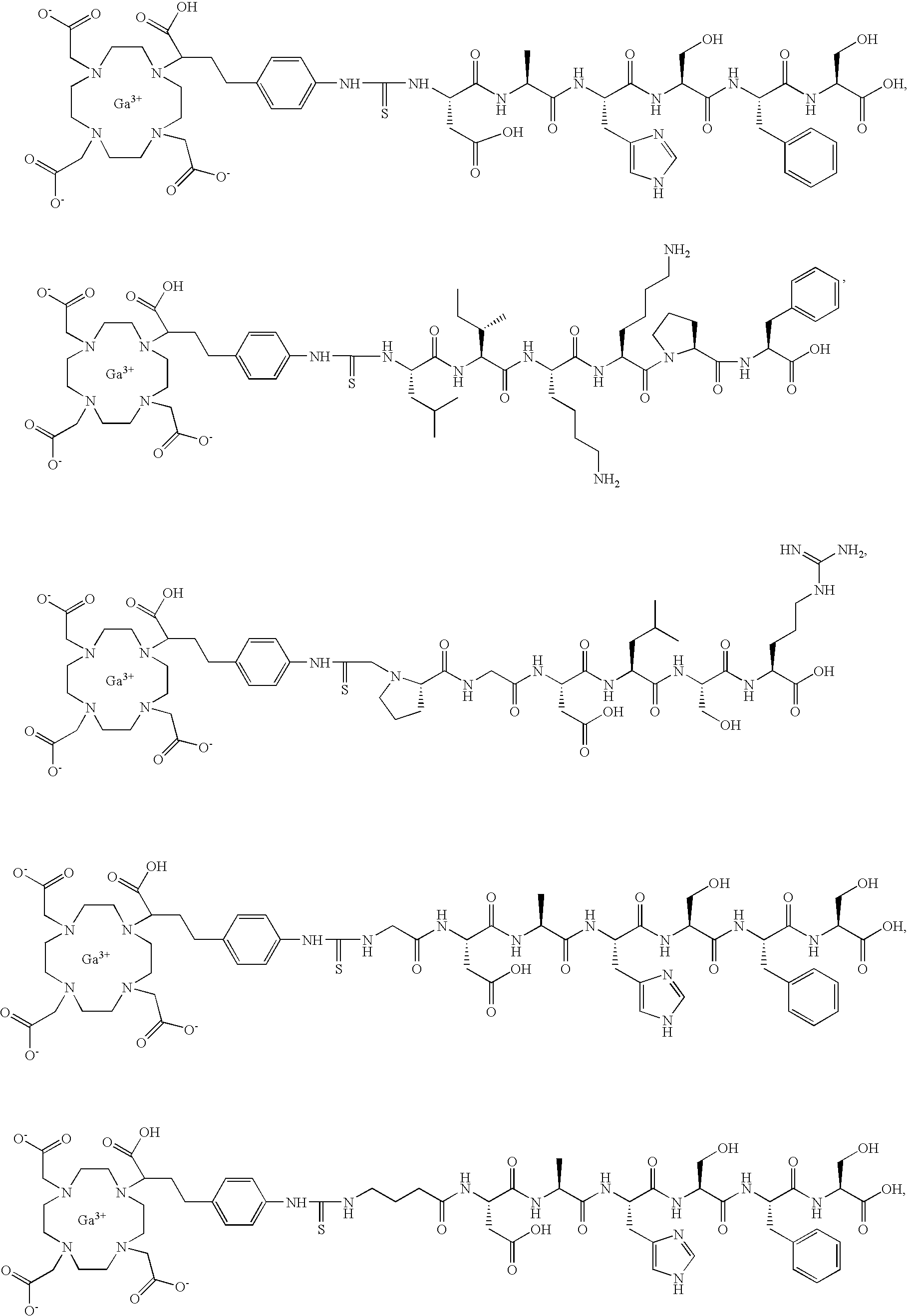

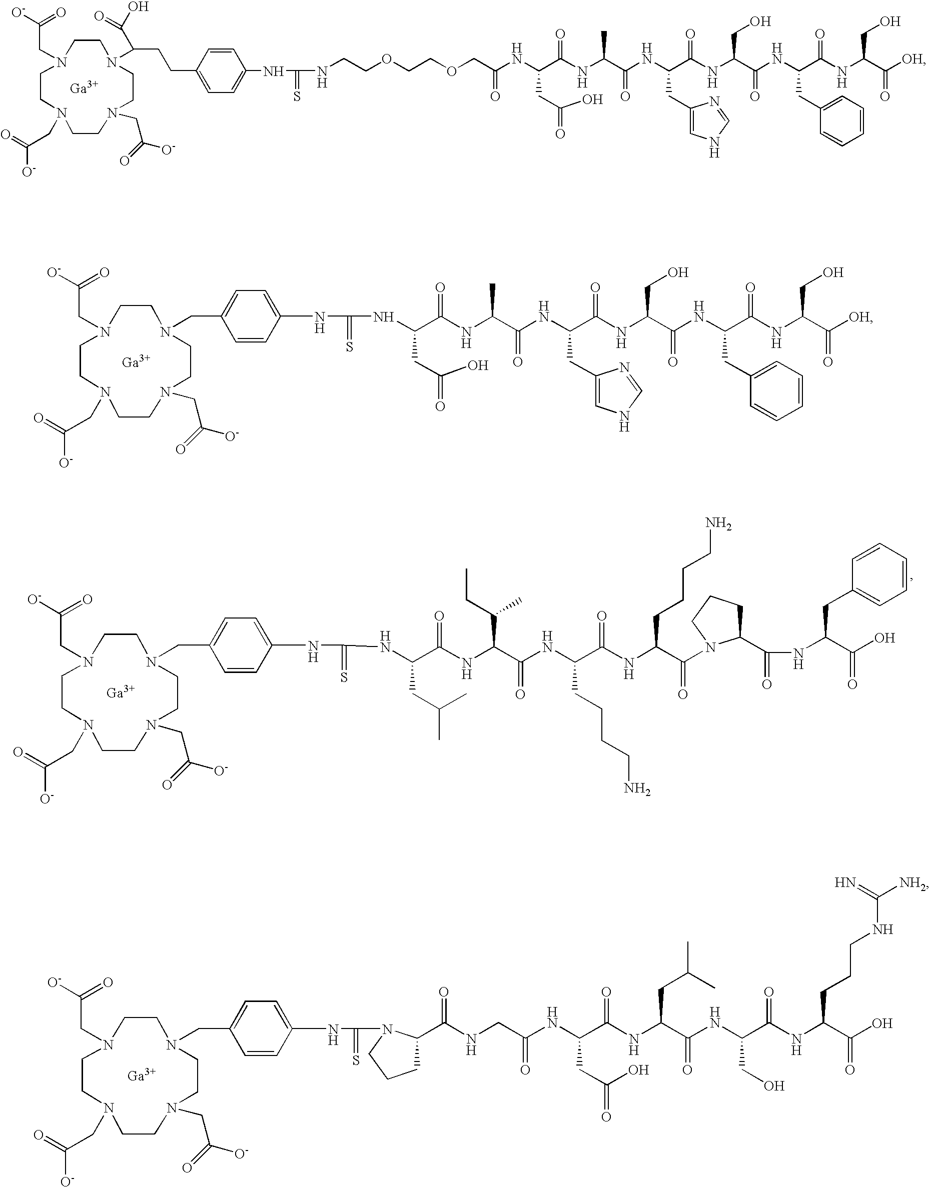

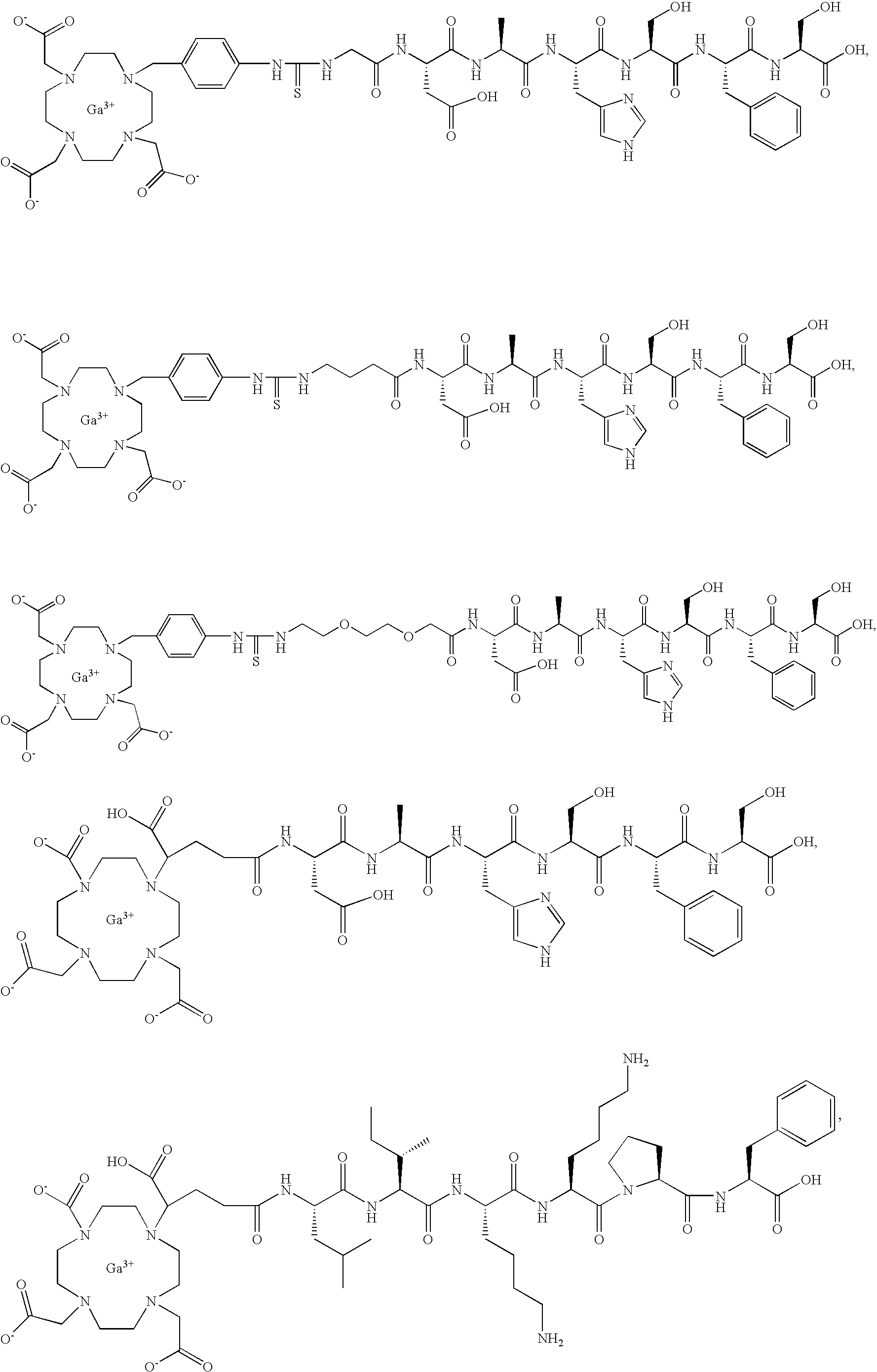

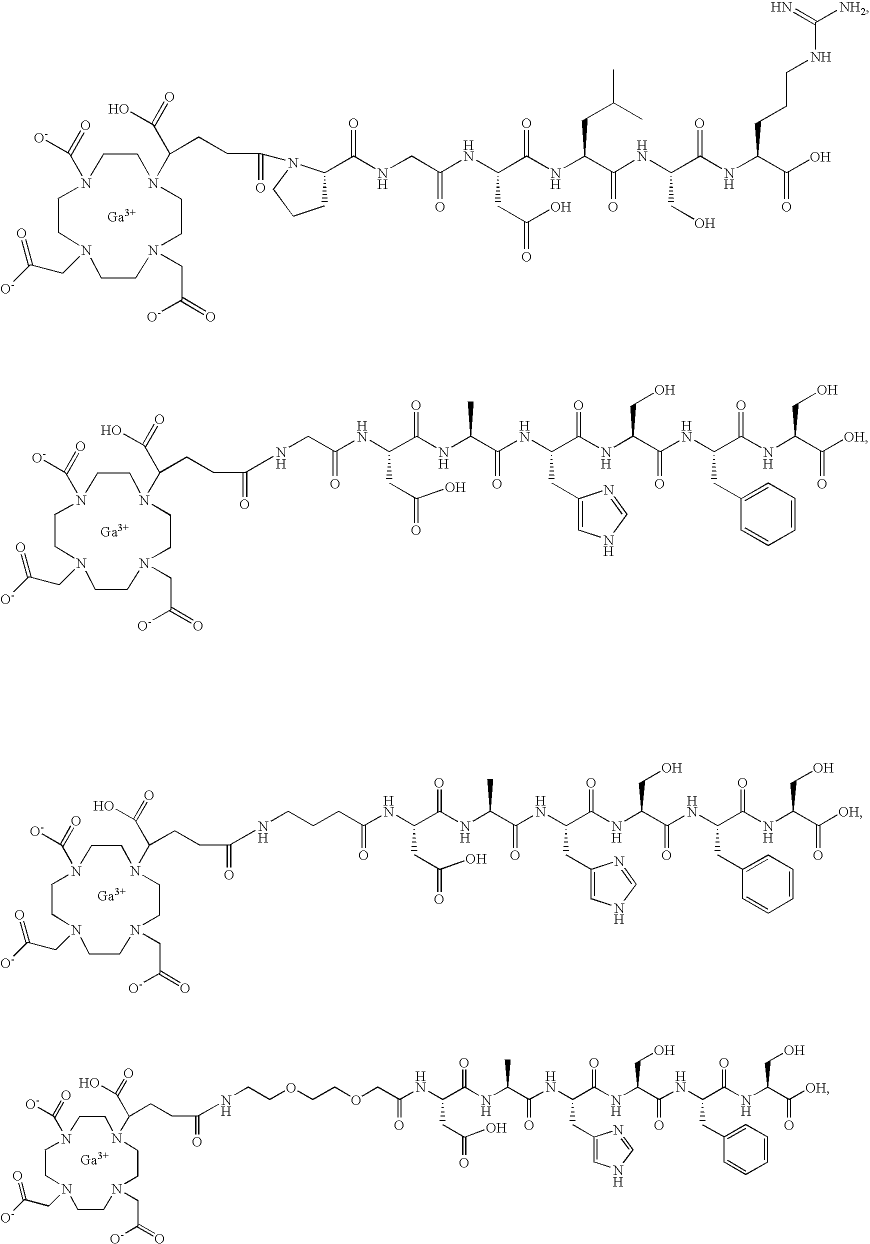

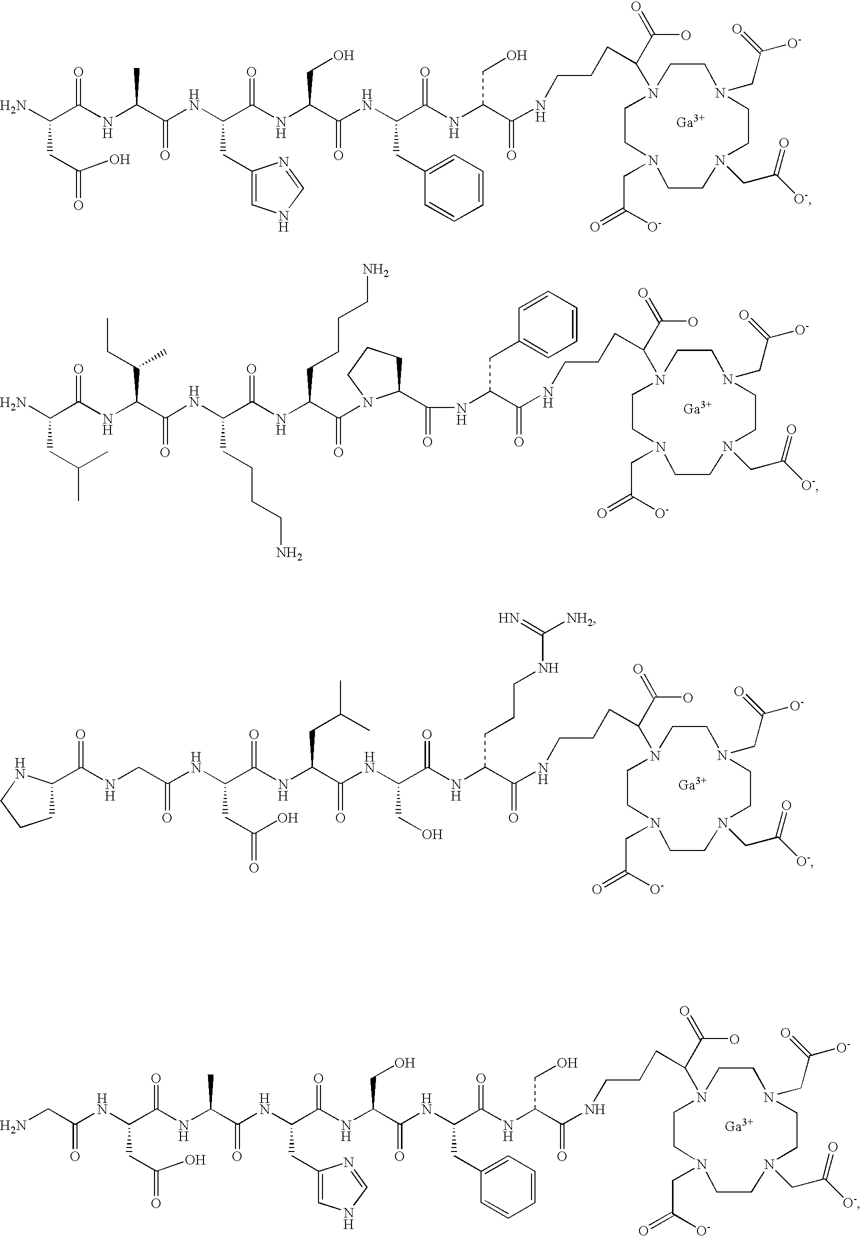

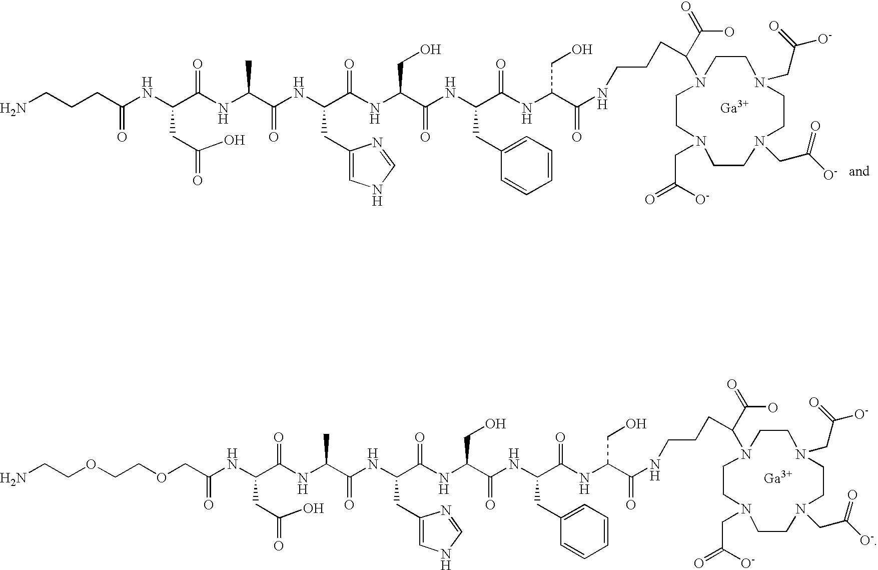

This is in particular the case of the compounds of formula I in which Signal represents a chelate coupled to a radionucleide, and in particular to the Ga68 radionucleide, particularly in the context of PET imaging.

According to one embodiment, the signal entity comprises at least one chelate Ch (in a form complexing a metal M). Advantageously, the chelate is coupled to the metal M. Advantageously, the metal M is a paramagnetic-metal ion, or a radionucleide. Advantageously, it is a paramagnetic metal. The complex formed by the chelate and the metal M is stable under physiological conditions so as to avoid undesired release of the metal M in the body. Advantageously, the chelate or the signal entity comprises at least one functional group for linking the signal entity to the Linker or directly to the Peptide. The invention also relates to the compounds of formula (I) in which the chelate is not complexed with the metal.

Advantageously, Ch is a linear chelate chosen from: EDTA, DTPA diethylenetriaminopentaacetic acid, N-[2-[bis(carboxymethyl)amino]-3-(4-ethoxy-phenyl)propyl]-N-[2-[bis(carboxymethyl)amino]ethyl]-L-glycine (EOB-DTPA), mono amide or bis amide derivatives of DTPA, such as N,N-bis[2-[carboxy-methyl[(methylcarbamoyl)methyl]amino]ethyl]glycine (DTPA-BMA), or 4-carboxy-5,8,11-tris(carboxymethyl)-1-phenyl-2-oxa-5,8,11-triazatridecan-13-oic acid (BOPTA).

Advantageously, Ch is a macrocyclic chelate chosen from 1,4,7,10-tetraazacyclododecan-1,4,7,10-tetraacetic acid (DOTA), 1,4,7,10-tetraazacyclododecan-1,4,7-triacetic acid (DO3A), 10-(2-hydroxypropyl)-1,4,7,10-tetraazacyclododecan-1,4,7-triacetic acid (HPDO3A), 2-methyl-1,4,7,10-tetraazacyclododecan-1,4,7,10-tetraacetic acid (MCTA), (alpha, alpha′, alpha″, alpha′″)-tetramethyl-1,4,7,10-tetraazacyclododecan-1,4,7,10-tetraacetic acid (DOTMA), 3,6,9,15-tetraazabicyclo[9.3.1]pentadeca-1(15),11,13-triene-3,6,9-triacetic acid (PCTA), 1,4,7-triazacyclononane-N,N′,N44-triacetic acid (NOTA), AAZTA (described in particular in WO 2006/00273, formula III, page 120 and US 2006/00118830, pages 2 and 89), TETA, TETMA, PDTA, and their benzo derivatives, LICAM, MECAM and HOPO (DE 102004062258).

Ch may also be a derivative of these compounds, in which one or more carboxylic groups is (are) in the form of a corresponding salt, ester or amide; or a corresponding compound in which one or more carboxylic groups is (are) replaced with a phosphonic and/or phosphinic group.

Use may also be made of a chelate chosen from: DOTA gadofluorines, DO3A, HPDO3A, TETA, TRITA, HETA, DOTA-NHS, M4DOTA, M4DO3A, PCTA and their derivatives, advantageously chosen from: DOTA, DTPA, DO3A, HPDO3A, TRITA, TETA, BOPTA, NOTA, PCTA, DOTMA, AAZTA, HOPO and their derivatives.

More broadly, the chelate(s) forming the signal entity may correspond to the following formula of document WO 01/60416:

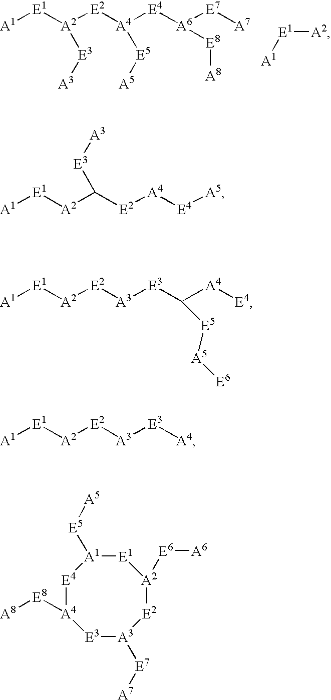

Use may in particular be made of the compounds DTPA, DOTA, NOTA, DO3A, and derivatives. Mention will also be made of the chelates recalled in WO 03/011115, in particular having the formulae below:

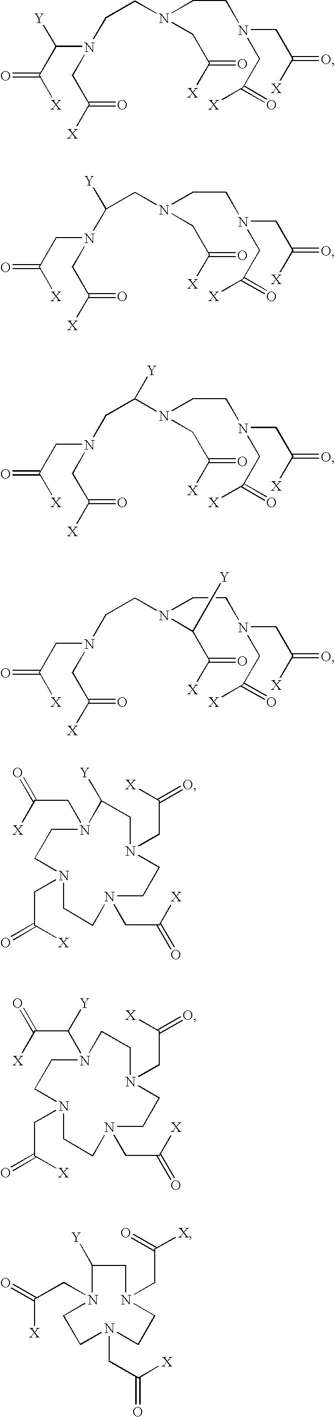

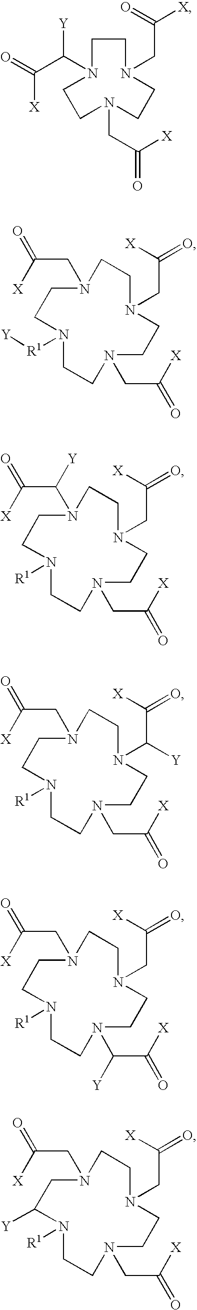

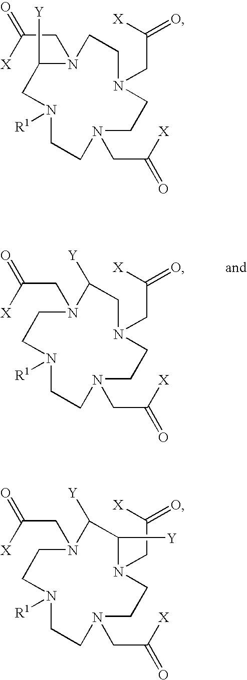

with X being a group capable of coordinating a metal cation, preferably O—, OH, NH₂, OPO₃— or NHR with R being an aliphatic chain, and Y a chemical linker.

Use may in particular be made of the chelates denoted P730, of the applicant, described in EP 661 279 (U.S. Pat. No. 5,919,432), having the formula:

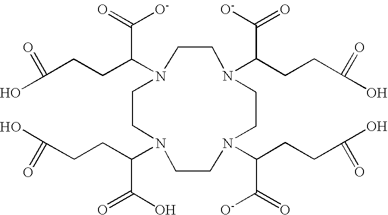

and the chelates with a PCTA backbone, described by the applicant in particular in U.S. Pat. No. 6,440,956, whether or not these chelates or their intermediates carry hydrophilic chains, and in particular short or long amino alcohol chains.

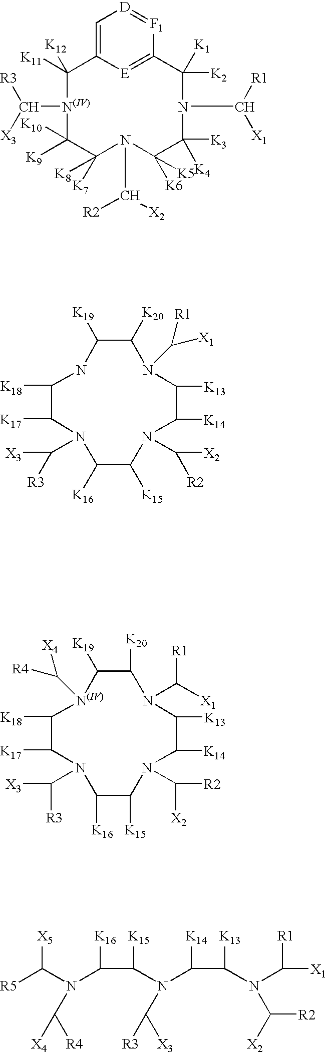

with X1 to X4 and K1 to K16 of the above chelates representing H or a C₁-C₂₀ chain, and R1, R2, R3, R4, R5 independently representing —COOH or —P(O)(OH)₂; and being chosen such that the chelate comprises at least one function capable of being coupled to a PEPTIDE P directly or by means of the Linker.

Mention will also be made of the chelates of document US2006/0018830, pages 9 to 11 of the description section [0150] to [0158].

Advantageously, in the context of the present invention, Ch is DTPA or DOTA or their derivatives.

In the case of MRI, the relaxivity r₁ of these chelates is typically of the order of 4 to 20 s⁻¹ mMol⁻¹ Gd⁻¹ with a field of 0.5 to 1.5 T. It is recalled that the longitudinal relaxivity r₁ of a paramagnetic contrast product gives the measure of its magnetic effectiveness and makes it possible to assess its influence on the recorded signal. In MRI medical imaging, the contrast products modify the proton relaxation time, and the increase in relaxivity obtained makes it possible to obtain a higher signal.

In formula I, the term “chemical bond” is intended to mean a linking group or Linker L, i.e. a chemical group:
which makes it possible to link the Signal and the PEPTIDE P(s),
which does not itself have the signal entity function that is provided by the Signal,
which does not itself have the targeting function that is provided by the PEPTIDE P.

The coupling of chelates with biovectors, in particular peptides, is described in the prior art, and generally involves a chemical bond (Linker) as described in document WO 01/60416. The structure and the chemical nature of the chemical bond are defined so as to enable chemical coupling between the peptide portion of the PEPTIDE P and the chelate(s) used, and in such a way as to obtain an affinity of the PEPTIDE P portion for its target and a specificity of recognition suitable for the use.

A large number of Linkers can be used, in so far as they are capable of interacting with at least one biovector functional group and at least one chelate functional group.

Advantageously, Linker represents:
a) a group of formula Q1-1-Q2,
in which Q1 and Q2, which may be identical or different, represent O, S, NH, CO₂, —NHCO, CONH, NHCONH, NHCSNH, SO₂NH— or NHSO₂—,
and 1 represents an alkyl group (advantageously C₁-C₁₀), alkoxyalkyl group (advantageously C₁-C₁₀), alkenyl group (advantageously C₂-C₆), alkynyl group (advantageously C₂-C₆), polyalkoxyalkylene group, alkyl group interrupted with one or more squarates, with one or more aryls, advantageously phenyl, or with one or more groups chosen from —NH—, —O—, —CO—, —NH(CO)—, —(CO)NH—, —O(CO)—, or —(OC)O—;
b) a (CH₂)ₙ, (CH₂)ₙ—CO—, —(CH₂)ₙNH—CO—, where n=2 to 10, (CH₂CH₂O)_{q}(CH₂)ᵣ—CO—, (CH₂CH₂O)q(CH₂)ᵣ—NH—CO— where q=1-10 and r=2-10, (CH₂)ₙ—CONH—, (CH₂)ₙ—CONH—PEG, (CH₂)ₙ—NH—,

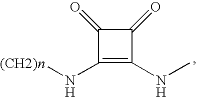

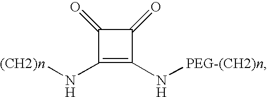

where n=1 to 5 and advantageously n=4 or 5, HOOC—CH₂—O—(CH₂)₂—O—(CH₂)₂—O—CH₂—COOH; HOOC—(CH₂)₂—CO₂—(CH₂)₂—OCO—(CH₂)₂—COOH; HOOC—CH(OH)—CH(OH)—COOH; HOOC—(CH₂)ₙ—COOH; NH₂—(CH₂)ₙ—NH₂, where n=1-20; NH₂—(CH₂)ₙ—CO₂H; or NH₂—CH₂—(CH₂—O—CH₂)ₙ—CO₂H, where n=1 to 10, group.
Among the advantageous linkers, mention will in particular be made of those of the examples of the present application:

CH2-phenyl-NHCSNH-(CH₂O)_{I=1 to 3}

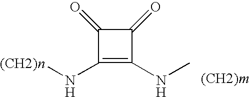

where n=1 to 5 and m=0 to 5

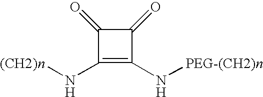

where n=1 to 5 and m=0 to 5 (CH2)n-CO where n=1 to 5 (CH₂)₃-squarate-(CH₂CH₂O)₂(CH₂)—CO;
c) linkers described in U.S. Pat. No. 6,264,914, capable of reacting with amino, hydroxyl, sulfhydryl, carboxyl, carbonyl, carbohydrate, thioether, 2-aminoalcohol, 2-aminothiol, guanidinyl, imidazolyl, phenolic functional groups (of the biovector and of the chelate); according to the definitions of this document;
d) certain linkers described in U.S. Pat. No. 6,537,520, of formula:
   (Cr₆r₇)_{g}-(W)ₕ-(Cr₆ₐr₇ₐ)_{g′}-(Z)ₖ-(W)_{h′}-(Cr₈r₉)_{g″}-(W)_{h″}-(Cr₈ₐr₉ₐ)_{g′″} where: g+h+g′+k+h′+g″+h″+g′″ is other than zero; with the definitions identical to those of this document, column 8;
e) certain linkers described in document WO 02/085908 (with the definitions identical to those of this document), for example a linear or branched organic linking chain chosen from:
   CR6′″R7′″-, —(R6′″)C═C(R7′″)═, —CC—, —C(O)—, —O—, —S—, —SO₂—, —N(R3′″)—, —(R6′″)C═N—, —C(S)—, —P(O0(OR3′″)—, —P(O)—(OR3′″)O—, where R′″3 is a group capable of reacting with a nitrogen or an oxygen,
   a cyclic region (divalent cycloalkyls, divalent heterocycles),
   polyalkylenes, polyalkylene glycols;
f) linkers of document WO 03/011115, pages 124-125;
g) linkers of document US 2006/0018830 (the Linker of the applicant corresponding to the linker denoted N—O—P in this document US 2006/0018830),
   linkers comprising at least one non-alpha amino acid (pages 12 to 15, table 1 of this document),
   linkers comprising at least one non-alpha amino acid carrying a cyclic group (pages 18 to 25, table 3 of this document),
   linkers not comprising an amino acid,
   other linkers (pages 27, 28 of this document).

The choice of Linker (structure and size) may be carried out in particular in such a way as to control especially the charge, the lipophilicity and/or the hydrophilicity of the product of formula (I), so as to optimize the biological targeting, the biodistribution. Linkers that are biodegradeable in vivo, PEG linkers or mini-PEG linkers may in particular be used. The linker is chosen in such a way as not to detrimentally alter the effectiveness of the compound of formula (I) according to the invention, a test for verifying this effectiveness in vitro and in vivo being present in the detailed description.

According to another embodiment, Signal represents a label for optical imaging (fluorescent molecule used in optical imaging). Among the labels for optical imaging, mention will in particular be made of those of US2006/0018830, and in particular those cited on pages 11 and 12, paragraph 1.B, with precise imaging modes described in column 33 ([0259]) in paragraph 6 (techniques and chromophores and fluorophores described in detail).

According to another embodiment, Signal represents quantum dots (inorganic fluorophores comprising nanocrystals).

According to another embodiment, Signal represents a superparamagnetic nanoparticle coated with an organic layer, advantageously commonly denoted SPIO or USPIO (“ultra small particles of iron oxide”). Advantageously, the nanoparticle comprises a core of iron oxide or hydroxide, in particular of magnetite (Fe₃O₄), maghemite (γ-Fe₂O₃). Use will advantageously be made of a nanoparticle covered with a bisphosphonate, advantageously gem-bisphosphonate, coating, described in WO2004058275, the particle and the method for coupling between the peptide and the nanoparticle being described in detail in the examples of the present application. The magnetic nanoparticles used are acidic nanoparticles based on an iron compound, and covered with a layer comprising one or more gem-bisphosphonate compounds, which may be identical or different, the nanoparticle-covering layer having the formula (C) below:

T-L2-CH(PO₃H₂)₂ (C)

in which:
the linker L2 represents an organic group linking the function T to the gem-bisphosphonate —CH(PO₃H₂)₂ function;
T represents a chemical function coupled to the PEPTIDE P or to the Linker of the present application. In one particular embodiment, T-L2 represents the Linker of the compound of formula (I).

The composition is in the form of a stable aqueous solution of nanoparticles. In these compositions, the degree of complexation (of the layer with the peptide) of the compound (C) on the particles is greater than 50%, advantageously than 70%, and preferably greater than 80, 90, 95%. It is particularly preferred for the acidic magnetic particles (p) to be complexed on at least 90% of their protonated sides with compounds of formula (C). According to one variant, a part of the functions T of the layer is coupled to a PEPTIDE P, and a part of the functions T is coupled to a hydrophilic compound, in particular a compound carrying hydroxyl groups, and in particular an aminoalcohol hydrophilic compound denoted AAG1AA28, described in WO2004058275 (example 8), or a PEG group.

The magnetic particles (p) have a hydrodynamic diameter of between 5 and 300 nm, preferably between 5 and 60 nm, more preferably between 5 and 30 nm.

The linker L2 makes it possible to link and/or to space out the gem-bisphosphonate function and the reactive entity T capable of providing the covalent grafting of the PEPTIDE P (the biovector) onto the nanoparticle, possibly by means of the Linker.

By way of preference, the linker L2 represents a divalent group.

Preferably, the linker L2 is chosen from:
an aliphatic group; alicyclic group; aliphatic alicyclic group; aromatic group; aliphatic aromatic group, it being possible for said aliphatic, alicyclic and aromatic groups to be optionally substituted with a methyl, hydroxyl, methoxy, acetoxy or amido group, or a halogen atom, advantageously a chlorine, iodine or bromine atom;
an -1₁-NHCO-1₂ group where 1₁ and 1₂, which may be identical or different, represent an aliphatic group; alicyclic group; aromatic group; aliphatic alicyclic group or aliphatic aromatic group, it being possible for said groups to be optionally substituted with a methyl, hydroxyl, methoxy, acetoxy or amido group, or a chlorine, iodine or bromine atom.

According to preferred embodiments, L2 represents a substituted or unsubstituted aliphatic group, and more preferably a —(CH₂)ₚ— group, where p is an integer from 1 to 5, or preferably a —(CH₂)ₙ—NHCO—(CH₂)₁ₙ— group where n and m represent an integer from 0 to 5.

By way of preferred T groups, mention may in particular be made of COOH, —NH₂, —NCS, —NH—NH₂, —CHO, alkylpyrocarbonyl (—CO—O—CO-alk), acylazidyl (—CO—N₃), iminocarbonate (—O—C(NH)—NH₂), vinylsulfuryl (—S—CH═CH₂), pyridyldisulfuryl (—S—S-Py), haloacetyl, maleimidyl, dichlorotriazinyl and halogen groups, particular preference being given to —COOH and —NH₂ groups.

Preferably, T represents a —COOH or —NH₂ group and L2 a substituted or unsubstituted aliphatic group advantageously a —(CH₂)ₚ— group, where p is an integer from 1 to 5.

The layer of formula (C1) below:

HOOC—(CH₂)₂—CH(PO₃H₂)₂

is most particularly preferred.

Several compounds of the nanoparticles of iron oxide carrying PEPTIDE P type are described in the detailed description.

According to another less advantageous embodiment, Signal represents a lipid nanoparticle comprising at least one chelate. The lipid nanoparticles may be in the form of a nanoparticulate emulsion, possibly containing perfluorocarbons, such as those described in documents WO 03/062198, U.S. Pat. No. 5,958,371, U.S. Pat. No. 5,080,885 and U.S. Pat. No. 6,403,056. The lipid nanoparticles may be suspended in an aqueous or hydrophilic medium. These nanoparticles have a diameter of the order of 10 nm to 500 nm, in particular 20 to 250 nm. The nanoparticles in emulsion may comprise or be coupled with a large number of chelates, for example 10 000 to 100 000 chelates per nanoparticle. The emulsions comprise sufficient compounds of formula (I) and therefore of Peptide to allow recognition of the region of apoptosis. As possible nanoparticles, mention will be made of liposomes, which may be unilamellar or multilamellar, micelles, microgels, oil droplets, lipoproteins, such as HDL, LDL, IDL or VLDL, chylomicrons, fluorocarbon nanoparticles, nanobubbles, or the like, the surface of which is lipophilic. Advantageously, the chelate is lipophilic and attached to the membrane of the nanoparticle.

Advantageously, the Linker of the compound of formula (I) is sufficiently lipophilic for coupling the Peptide to the membrane of the lipid nanoparticle, the PEPTIDE P being sufficiently expressed on the outer part of the nanoparticle for specific recognition of the apoptotic target. The Linker is, for example, a lipophilic group such as a C₁₀-C₂₀ alkylene chain, this chain being inserted into the lipid layer of the nanoparticle and thus making it possible to attach the Peptide to the nanoparticle.

Many chelates made lipophilic so as to be associated with a lipid membrane are described in detail, in particular in documents U.S. Pat. No. 6,045,821, WO 90/04943 and WO 2006/100305. Depending on embodiments, the chelate carries a long lipophilic chain (phospholipid, for example) which is inserted into the membrane of the lipid nanoparticle (liposome, micelle, nanoemulsion). Similarly, the PEPTIDE P advantageously carries a lipophilic chain (the Linker) which is inserted into the membrane of the lipid nanoparticle.

According to advantageous embodiments, the lipid nanoparticle includes perfluorocarbons as described in U.S. Pat. No. 5,958,371, the liquid emulsion containing nanoparticles comprising a perfluorocarbon having quite a high boiling point (for example, between 50 and 90° C.), surrounded by a coating composed of a lipid and/or of a surfactant. The surfactant is capable of coupling directly to a targeting biovector or of including an intermediate compound covalently bonded to the biovector, where appropriate, by means of a chemical bonding agent.

Various perfluorocarbon emulsions are recalled in document U.S. Pat. No. 6,676,963 (perfluorodecalin, perfluorooctane, perfluorodichlorooctane, perfluoro-n-octyl bromide, perfluoroheptane, perfluorodecane, perfluorocyclohexane, perfluoromorpholine, perfluorotripropylamine, perfluorotributylamine, perfluorodimethylcyclohexane, perfluorotrimethylcyclohexane, perfluorodicyclohexyl ether, perfluoro-n-butyltetra-hydrofuran).

As phospholipids forming the membrane of the nanoparticles, use is customarily made of the following compounds: phosphatidylcholine, dioleoylphosphatidylcholine, dimyristoyl phosphatidylcholinedipalmitoylphosphatidylcholine, distearoylphosphatidylcholine, phosphatidylethanolamine.

Such compositions are described, for example, in U.S. Pat. No. 5,989,520 and U.S. Pat. No. 5,958,371, as recalled in document US 20040248856 which in particular cites perfluorocarbon compounds: perfluorodecaline, perfluorooctane, perfluorodichlorooctane, perfluoro-n-octyl bromide, perfluoroheptane, and the like.

According to advantageous embodiments, in order to prepare contrast agents according to the invention, use will be made of appropriate methods and lipid compositions recalled in U.S. Pat. No. 6,010,682, in particular as regards the detailed description of the lipid composition, and of the preparation of liposomes, of micelles and of emulsions.

It is recalled that emulsions are heterogenous lipid mixtures obtained in an appropriate manner by mechanical stirring and/or addition of emulsifying agents. For example, the chelates rendered lipophilic are mixed mechanically with organic solvents such as chloroform. After the solvent has been evaporated off, the lipids are resuspended in an aqueous medium such as PBS, so as to obtain an emulsion which then typically undergoes sonication and microfluidization. The emulsions obtained can be lyophilized with, where appropriate, the use of anti-agglutination agents.

Typically, 1% to 75% by weight of lipophilic chelate compound, relative to the total ingredients of the emulsion, are used to formulate the desired paramagnetic contrast agent emulsion. The composition forming the contrast agent is preferably administered intravascularly, depending on the patient examined, for example at a rate of 0.1 mg to 1 g of lipophilic chelate compound and of 1 to 50 micromol of paramagnetic-metal ion per kg of patient.

The lipid compositions obtained are, where appropriate, formulated using additives recalled in U.S. Pat. No. 6,010,682, in particular for administration by intraveinous injection. Mention will in particular be made of dextrose, sodium chloride and antimicrobial agents. Advantageously, by virtue of the compositions according to the invention, an increase in relaxivity per ion can be obtained. The following characteristics, which can vary depending on the precise compositions of the emulsions and the method for the preparation thereof, are typically obtained:
polydispersity index: 0.2 to 0.3
[Gd³⁺]=2 to 10 mM, preferably 3 to 7 mM
particle concentration: 50 to 100 nM
r1 (mM⁻¹s⁻¹Gd⁻¹): 5 to 40, preferably 10 to 40
r2 (mM⁻¹s⁻¹Gd⁻¹): 20 to 40
r1 (mM⁻¹s⁻¹ particle⁻¹): 10⁶ to 4×10⁶
number of biovectors: 50 to 1000, in particular 100 to 300.

The invention also relates to these compounds of formula (I) in which the Peptide contains a peptide sequence which has been modified, but without impairing the affinity and the specificity for the target and the effectiveness of the compound in vivo. The PEPTIDE P is, for example, modified using appropriate methods described in the prior art, for example in US2005100963 (column 20-21, paragraphs [529] to [541] in the case of peptides targeting KDR receptors), in order to select effective compounds of formula (I):

1) substitution of amino acids without impairing their function, according to the method recalled in this document for hydrophobic amino acids, aromatic amino acids, acidic amino acids, amino acids containing hydroxyls, amino acids containing amide side chains.

For example, for the hydrophobic amino acids leucine X1 and isoleucine X2, the use of another linear or branched C₁₋₁₀ aliphatic side chain (alkyl, alkenyl, alkynyl) may be tested. Substitution of the amino acids X1 and X2 with other alkyl-substituted hydrophobic amino acids (alanine, leucine, isoleucine, valine, norleucine, S-2-aminobutyric acid) may thus be tested.

For proline, other hydrophobic amino acids, in particular derivatives with a secondary amine, are also used.

For X6 (phenylalanine), the use of other aromatic amino acids (tyrosine, biphenylalanine, naphthylalanine, benzothienylalanine, and derivatives) is tested. The derivatives of paragraph [532] of US2005100963, in particular the amino-, alkylamino-, dialkylamino- and aza-substituted derivatives, halogenated derivatives and alkoxy-substituted derivatives, such as 2-, 3- or 4-aminophenylalanine or 2-, 3- or 4-methylphenylalanine, may in particular be screened.

For X3 and X4 (lysine), other dibasic amino acids (arginine, histidine, ornithine) or derivatives of lysine or of these other amino acids, in particular alkyl, alkenyl or aryl derivatives, such as N-epsilon-isopropyllysine derivatives, may also be used. The derivatives cited in paragraph [533] of US2005100963 may in particular be tested;

2) substitution of amide bonds present on the backbone of the polypeptide, in particular so as to limit degradation of the peptide and/or to adjust the flexibility of the peptide (for example, insertion of alpha-N-methylamide or of a thioamide, replacement of an amino acid with an aza-amino acid);

3) introduction of D-series amino acids, such as D-alanine, in order to adjust the accessibility of the peptide for its target owing to an effect of steric modification on the orientation of the side chains;

4) chemical modifications in order to adjust the solubility and pharmacokinetics of the compound of formula (I), for example by adding a hydrophilic or basic group, or an alkyl or aromatic nonpolar group, by means of bonding to the C- or N-terminal part of the peptide, or to an amino acid side chain of the peptide, in particular to lysine which has a free amine function (or a derivative such as 2-,3-diaminopropionic acid);

5) glycosylation;

6) other modifications:
formation of salts: N-methylglucamine (meglumine), acetate, oxalates, ascorbates, etc.,
manipulation of the peptide sequence (for example, retro-peptide or cyclization).

Thus, for the peptide D-A-H-S-X7-S (SEQ ID No 2), where advantageously X7 is chosen from F or L, a study is also made of the peptides (SEQ ID No 7) with:

A replaced with leucine, isoleucine, valine and derivatives,

H replaced with lysine or arginine and derivatives,

S replaced with threonine and derivatives,

L replaced with isoleucine, valine and derivatives.

Thus, for the peptide P-G-D-L-X8-X9 (SEQ ID No 3), or advantageously X8 is chosen from S or V and X9 is chosen from T or R, a study is also made of the peptides (SEQ ID No 8) with:

L replaced with isoleucine, valine and derivatives,

T replaced with serine,

R replaced with lysine,

D replaced with aspartic acid.

Thus, for the peptide H-G-X10-L-S-X11 (SEQ ID No 4), or advantageously X10 is chosen from D or H, and X11 is chosen from T or I, a study is also made of the peptides (SEQ ID No 9) with:

L replaced with isoleucine, arginine, valine and derivatives,

H replaced with lysine or arginine and derivatives,

G replaced with asparagine,

T replaced with swine,

I replaced with leucine, valine and derivatives.

Thus, for the peptide VLGERG (SEQ ID No 5), a study is also made of the peptides (SEQ ID No 10) with:

V replaced with leucine or isoleucine,

L replaced with isoleucine, arginine, valine and derivatives,

E replaced with aspartic acid,

R replaced with lysine.

The biological tests described in detail in the application make it possible to select effective peptides which are equivalent or improved in terms of apoptosis-targeting activity in comparison with the peptides exemplified in detail in the present application.

According to another aspect, the invention relates to the MRI contrast products comprising a compound of formula (I) as described above, the paramagnetic-metal M ion of which has the atomic number 21-29, 42-44 or 58 or 70, preferably gadolinium. The paramagnetic metals M include lanthanides having the atomic number 58-70 and transition metals having the atomic number 21-29, 42 or 44, for example scandium, titanium, vanadium, chromium, manganese, iron, cobalt, nickel, copper, molybdenum, ruthenium, cerium, praseodymium, neodymium, promethium, samarium, europium, gadolinium, terbium, dysprosium, holmium, erbium, thulium and ytterbium. The elements Gd(III), Mn(II), europium and dysprosium are particularly preferred; advantageously, M is chosen from Gd, Mn, Fe, Dy and Tm.

According to another aspect, the invention relates to the contrast products for X-ray or CT imaging, comprising a compound (I) as defined above, the heavy metal M ion of which has the atomic number 21-31, 39-50, 56-80, 82, 83 or 90.

According to another aspect, the invention relates to radiopharmaceutical products comprising a compound of formula (I) as described above, the chelate of which is coupled with a radionucleide or a radiohalogen known to those skilled in the art, typically gadolinium, technetium, chromium, gallium, indium, ytterbium, rhenium, lanthanum, yttrium, dysprosium, copper, or the like. The radionucleides include the radioactive forms of the elements Sm, Ho, Y, Pm, Gd, La, Lu, Yb, Sc, Pr, Tc, Re, Ru, Rh, Pd, Pt, Cu, Au, Ga, In, Sn, Cr, Pb, in particular ⁹⁹Tc, ¹¹⁷Sn, ¹¹¹In, ⁹⁷Ru, ⁶⁷Ga, ⁶⁸Ga, ₈₉Zr, ¹⁷⁷Lu, ⁴⁷Sc, ¹⁰⁵Rh; ¹⁸⁸Re, ⁶⁰Cu, ⁶²Cu, ⁶⁴Cu, ⁶⁷Cu, ⁹⁰Y, ¹⁵⁹Gd, ¹⁴⁹Pr, ¹⁶⁶Ho; ⁶⁸Ga is the least advantageous in the context of the present invention. The preparation of compounds that can be used as radiopharmaceuticals, in particular using technetium ⁹⁹Tc, is recalled in US 2006/0018830, page 32, paragraph 4, these techniques being described in this document for compounds comprising a chelate coupled to a gastrin-targeting peptide. For indications in radiotherapy, the coupling of macrocycles of DOTA type, the selection of appropriate nucleides and the preparation of the radiotherapeutic compounds are recalled in US 2006/0018830, columns 35 and 36.

According to another aspect, the invention relates to a method of diagnosis and a method of radiopharmaceutical treatment using a compound of formula (I) as described above.

The present invention also relates to a composition comprising at least one compound of general formula (I) as described above and a pharmaceutically acceptable excipient, advantageously for parenteral administration. In addition, it relates to a method for preparing such a composition, comprising the addition of a compound of general formula (I) as defined above to an injectable medium comprising the pharmaceutically acceptable excipient.

The invention relates to the use of a composition according to the present invention, for the diagnosis of a pathological condition associated with apoptosis. The diagnostic and radiopharmaceutical compositions according to the invention can be used as described in applications US 2002/0090342, US 2002/0098149, WO 02/055111 for anticancer indications.

The invention relates in addition to the compounds of general formula (I) as defined above, for their use as an agent for diagnosing diseases associated with apoptosis. The invention also relates to the use of the compounds described above, for the preparation of a diagnostic or radiopharmaceutical composition for uses in the diagnosis and/or treatment of diseases associated with apoptosis, advantageously chosen from chronic neurodegenerative diseases, ischemic diseases and inflammatory pathological conditions.

Finally, it relates to the use of an apoptosis-targeting peptide of formulae (1) to (5) as defined above, for the preparation of a composition for diagnosing diseases associated with apoptosis.

Where appropriate, the compounds of general formula (I) of the applicant will be used as a diagnostic agent, or as an agent for therapeutic treatment at regions of apoptosis, or as a diagnostic and therapeutic treatment agent, or an agent for diagnostic monitoring of the therapeutic effectiveness. Where appropriate, the compound will be administered simultaneously, or after a delay, with other apoptosis-targeting diagnostic and/or therapeutic agents. The invention also relates to an imaging method comprising the synthesis of a compound comprising a paramagnetic metal according to the invention, capable of targeting a pathological region, its administration to a patient, and imaging by MRI. The invention also relates to an imaging method comprising the synthesis of a radiopharmaceutical compound according to the invention, capable of targeting a pathological region, its administration to a patient, and imaging by SPECT or planar gamma scintigraphy, or positron emission tomography.

For a diagnosis by MRI, the intraveinous administration by injection, customarily in a saline solution, is typically carried out at a dose of metal ion of from 0.001 to 1.5 mmol/kg of body weight, for example from 1 to 500 nmol Gd/kg.

For a radiopharmaceutical diagnosis, the intraveinous administration by injection, customarily in a saline solution, is typically carried out at a dose of from 1 to 100 mCi per 70 kg of body weight, preferably from 5 to 50 mCi, with diagnostic imaging, for example, 30 to 180 minutes after the injection for ⁹⁹Tc.

For use as X-ray contrast agents, the concentration of heavy atom is typically from 0.1 M to 5 M, with concentrations by intraveinous administration of the order of 0.5 to 1.5 mmol/kg.

According to another aspect, the invention relates to the use of a compound of formula (I) as described above, for the preparation of a composition for use in optical imaging.

Examples of administration of compositions for medical imaging are described in the prior art, for example in document WO 0226776 and US 2006/0018830, column 36 ([0282]), paragraph 8 (dosages and additives).

Pharmaceutically, physiologically acceptable carriers that make it possible to form diagnostic compositions (contrast products) comprising the compounds described above are known from the prior art. Use will, for example, be made of salts (sodium, calcium, meglumine), pH regulators (acetic acid, citric acid, fumaric acid) and antioxidants.

The invention also relates to a method for preparing compounds, comprising the coupling of a Peptide with at least one chelate. Several general methods preparing compounds of formula (I) described in US2006/0018830 (Bracco) are applicable, with Peptide being used in place of the peptides of these documents. These methods selected according in particular to the selected chelate are recalled in 2006/0018830, column 37 ([0288] to ([0291]) (“general preparation of compounds” and “alternative preparation of the compounds via segment coupling”), for example the SPPS and FMOC methods.

The invention also relates to a radiopharmaceutical treatment or diagnostic method, which comprises administering a compound (I), carrying out an imaging examination using suitable equipment, and analyzing the results.

Unless otherwise indicated, the invention covers all the chiral, diastereoisomeric, racemic, in particular cis-trans, and L-D forms of the compounds of formula (I) described.

The applicant has also studied the possibilities of association of a PEPTIDE P coupled to several chelates in the compound of formula (I). The applicant has, moreover, studied compounds of formula (I) exhibiting an assembly between one or more Peptides of the compound of formula (I), targeting apoptosis, and one or more chelates, in such a way that the access to the target is not impaired despite the presence of the chelate(s). For example, the chelate is distanced from the PEPTIDE(S) P by the Linker of sufficient size and with a chemical structure such that the recognition of the peptide(s) by its (their) target is not impaired.

Among the biovector (peptides or optional other biovectors)/chelate associations, mention may in particular be made of:
a central biovector peptide linked to several chelates which may be identical or different;
a central chelate linked to several peptides which may be identical or different;
a first [peptide carrying chelate(s)] assembly coupled by means of a hydrophobic or hydrophilic linker to a second [peptide carrying chelate(s)] assembly, which is written, for example:

Ch2-peptide1-Linker-peptide2-Ch2 with Ch representing chelates which may be identical or different, and peptide1 and peptide2 representing Peptides which may be identical or different;
a peptide1-(Linker carrying Ch)-peptide2-Ch assembly.

Use will, for example, be made of the method for constructing multimeric compounds described in US2005/0100963 (W02006/031885, page 66, line 25 to page 69, line 30) in the case of peptides targeting KDR receptors (for example, by means of method 13 of the examples: “preparation of homodimers and heterodimers”), but using the PEPTIDE P (the peptides of the compounds of FIGS. 44 to 47 of US2005100963 will, for example, be replaced with PEPTIDES P). The compound may thus advantageously comprise a peptide coupled to several chelates, or a chelate coupled to several peptides, which may be identical or different. The invention also relates to mixed compounds comprising, in addition to the PEPTIDE P, at least one other apoptosis-targeting biovector, the biovector being either another peptide, or another biovector, but which is a nonpeptide biovector.

Where appropriate, the peptide portion or the chelate portion may be coupled to chemical groups which make it possible to promote the biodistribution and/or the lifetime of the product in the blood.

The specificity of the product refers to its specific affinity for at least one marker for apoptosis or for any associated pathological disorder, the binding specificity being expressed typically by Kd and Ka constants, the Kd value for the target markers being less than 10 μM, preferably less than 1 μM.

Among the pharmaceutically acceptable salts, mention will in particular be made of salts of cations of inorganic bases (potassium, sodium, calcium, magnesium, etc.), of cations of organic bases (ethanolamine, diethanolamine, morpholine, glucamine, N-methylglucamine, N,N-dimethylglucamine, etc.), of anions of inorganic acids (in particular chlorides, bromides, iodides, sulfates, etc.), of anions of organic acids (acetate, succinate, citrate, fumarate, maleate, oxalate, trifluoroacetate, etc.), and of ions of amino acids (taurine, glycine, lysine, arginine, ornithine, aspartic acid, glutamic acid, etc.).

The applicant has demonstrated the very advantageous use of compounds incorporating peptides according to the invention, in particular in MRI, for several categories of disorders caused by apoptosis: vascular disease (apoptosis in regions where there are atheroma plaques), impairment of hepatic tissues (hepatic apoptosis), impairment of nerve tissues (neuronal apoptosis responsible for brain diseases, including Parkinson's disease in particular).

It is recalled that there is a very important difference between the potantial effectiveness in vitro of a targeting biovector, and its real effectiveness in vivo once coupled to a signal entity.

As illustrated below, the applicant has demonstrated in particular the effectiveness of products for MRI comprising a chelate linked to the peptide P, in particular LIKKPF, and of products for MRI of the type USPIO (particle of iron oxide) linked to the peptide P, in particular LIKKPF.

Thus, even if a peptide was known from the prior art, the identification of its usefulness in a mechanism of apoptosis through its targeting, in particular of phosphatidylserine, among the extremely large number of possible biological targets, is far from evident. In addition, it is in no way evident that this identified biological target, of the coupled compounds (peptide P—signal entity), would make it possible to solve the technical problems solved by the applicant, in particular:
conservation of the in vivo affinity for the biological recognition site, despite the steric hindrence and the possible conformational modification in vivo owing to the coupling to a signal entity;
the possibility of chemical coupling with the signal entities; the coupling with PCTA derivatives in particular has meant that a dedicated chemical synthesis has had to be developed
the physicochemical stability in vivo, which constitutes a major limitation for many peptides
the ability to be recognized in imaging in vivo, in particular in MRI, a technique for which the level of sensitivity is close to 1000 times less than PET imaging.

Overall, it was therefore in no way obvious, for those skilled in the art:
on the one hand, to select the peptides which are the subject of the present invention,
on the other hand, that the products integrating these peptides be actually effective in vivo.

The detailed description which follows describes peptides of which the effectiveness has been shown with variant embodiments on various signal entities (PART I) and the biological results (PART II).

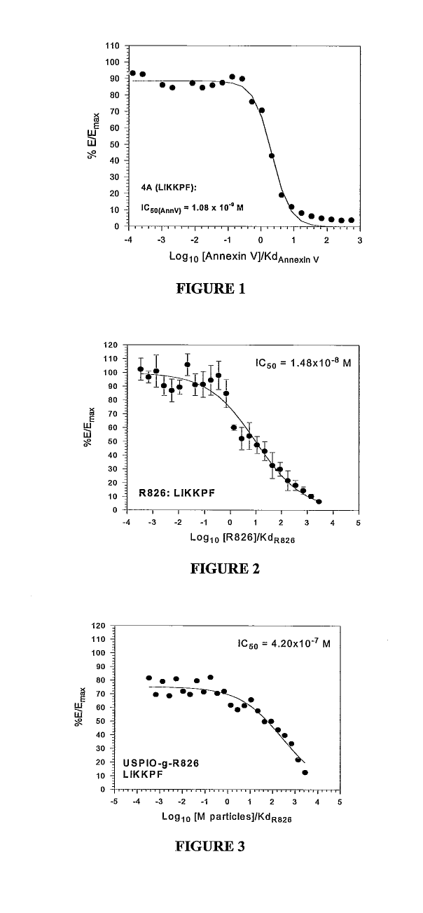

FIG. 1 represents the competition assay with annexin V.

FIG. 2 represents the measurement of the affmity of the peptide LIKKPF (SEQ ID No 11).

FIG. 3 represents the measurement of the affinity of the peptide LIKKPF (SEQ ID No 11) coupled to a USPIO.

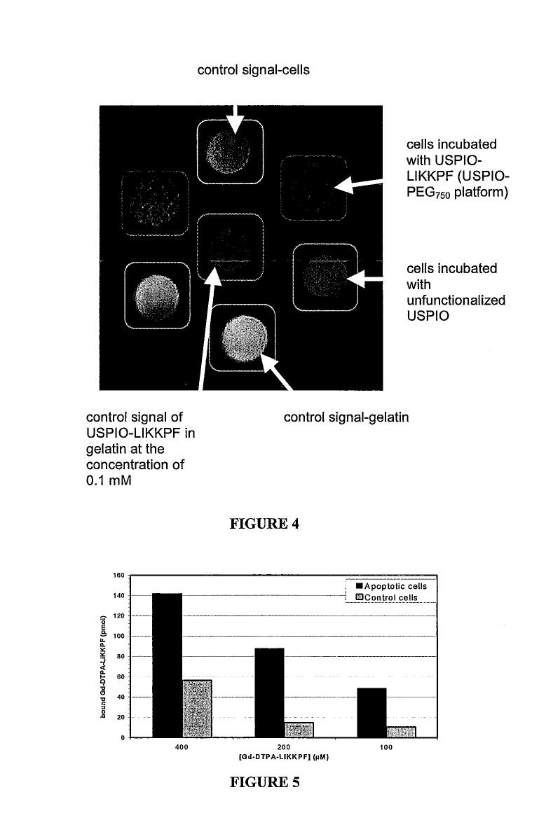

FIG. 4 illustrates tests on apoptotic cells carried out with a USPIO-LIKKPF product.

FIG. 5 illustrates the affinity of a DTPA-LIKKPF product with apoptotic cells.

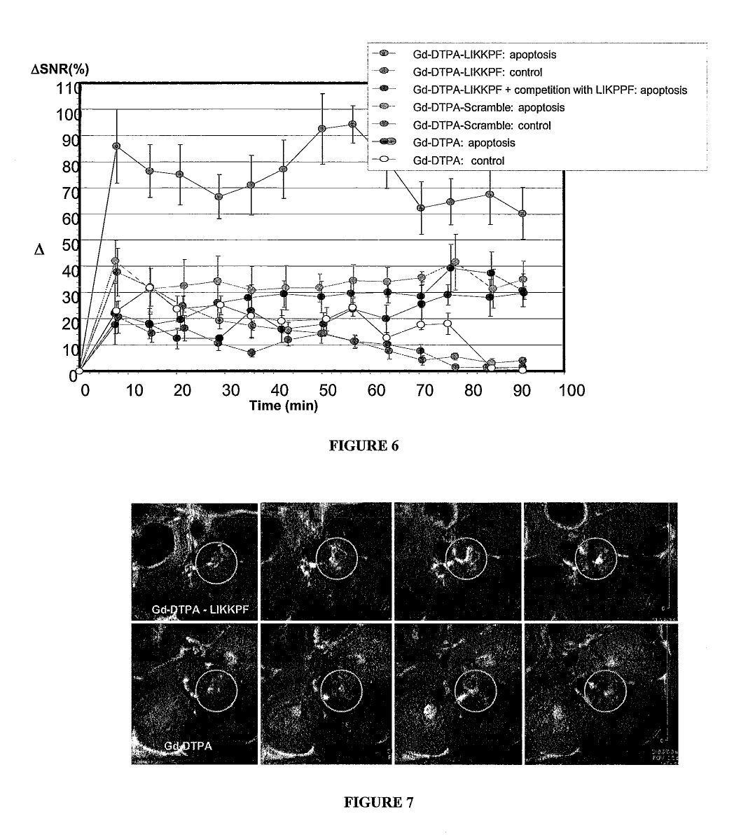

FIG. 6 illustrates the dynamic monitoring of the MRI signal of mouse apoptotic liver with a DTPA-LIKKPF product.

FIG. 7 shows MRI images on ApoE mice, obtained with a DTPA-LIKKPF product,

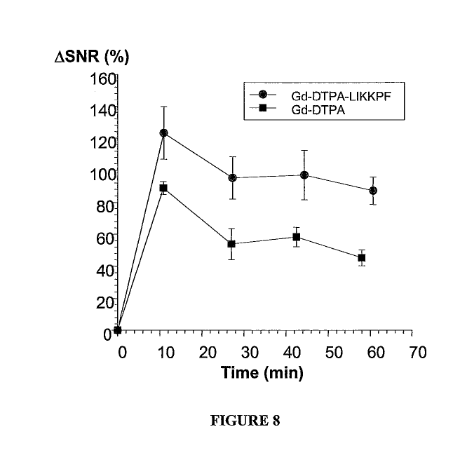

FIG. 8 represents the corresponding quantitative enhancement.

### EXAMPLES PART I

### Preparation of the Compounds Including PEPTIDES P

General Information
M: molar concentration (mol/l).
M/z: mass-to-charge ratio determined by mass spectrometry.
ES⁺: positive mode electrospray.
ES⁻: negative mode electrospray.
kDa: unit of molecular mass (kiloDalton).
TLC: Thin Layer Chromatography.
Zave: hydrodynamic diameter measured by PCS.

Assaying of Total Iron:

The iron is assayed by atomic absorption spectroscopy (VARIAN AA10 spectrophotometer) after mineralization with concentrated HCl and dilutation relative to a standard range of ferric ions (0, 5, 10, 15 and 20 ppm).

Particle Size:

Hydrodynamic Diameter of the Grafted Particle (Zave):

Determined by PCS (Malvern 4700 instrument, laser 488 mn at 90°) on a sample diluted to ˜1 millimolar with water for injectable preparationn, filtered through 0.22 μm.

PCS=Photon Correlation Spectroscopy=Technique by dynamic light scattering−Reference: R. Pecora in J. of Nano. Res. (2000), 2, p. 123-131.

Structural Analyses:

By mass spectroscopy (Micromass VG Quattro II instrument) with an electrospray source.

Relaxivity Measurements:

The relaxation times T1 and T2 were determined by standard procedures on a Minispec 120 instrument (Bruker) at 20 MHz (0.47T) and 37° C. The longitudinal relaxation time T1 is measured using an inversion-recovery sequence and the transverse relaxation time T2 is measured using a CPMG technique.

The relaxation rates R1 (=1/T1) and R2 (=1/T2) were calculated for various concentrations of total metal (ranging from 0.1×10⁻³ to 1×10⁻³ mol/l) in an aqueous solution at 37° C. The correlation between R1 or R2 as a function of the concentration is linear, and the slope represents the relaxivity r1 (R1/C) or r2 (R2/C) expressed as (1/second)×(1/mmol/l), i.e. (s⁻¹.mM⁻¹).

The nanoparticles were prepared according to the methods described in patent WO 2004/058275 (US 2004/253181), examples 8 and 9 for the preparation of colloidal solutions of magnetic particles, and examples 10 to 12 for the complexation of the magnetic particles with a gem-bisphosphonate coating of example 1 of WO 2004/058275.

### Example 1

### Coupling of the Peptides to the Particle of Iron Oxide

(Product PEG-USPIO-PEPTIDE P)

The peptide sequences of interest are given in the table below:

| No. | Sequence of the PEPTIDE P |
|---|---|
| 1 | Asp-Ala-His-Ser-Phe-SerOH |
| 2 | Leu-Ile-Lys-Lys-Pro-Phe-OH |
| | (SEQ ID No 11) |
| 3 | Pro-Gly-Asp-Leu-Ser-Arg-OH |
| 4 | Gly-Asp-Ala-His-Ser-Phe-SerOH |
| 5 | γ-Abu-Asp-Ala-His-Ser-Phe-SerOH |
| 6 | 8-Amino-3,6-dioxaoctanoyl-Asp-Ala-His-Ser-Phe- |
| | SerOH |
| 7 | 8-Amino-3,6-dioxaoctanoyl-Leu-Ile-Lys-Lys-Pro- |
| | Phe-OH |

Coupling of the Peptide:

30 ml of nanoparticles, [Fe]=0.338 mol, are ultrafiltered and stirred at ambient temperature, the pH is equal to 7.29. Peptide No. 2 (LIKKPF (SEQ ID No 11)), protected in trifluoroacetamide form on the side amine functions of the lysines, is dissolved in 1 ml of water (26.6 mg) and the solution obtained is gradually added at the same time as the EDCI (25 mg). Once the addition is complete, the pH is equal to 6.55. The mixture is stirred overnight at ambient temperature. The pH is adjusted to 7 with NaOH (0.1 N) and the solution is filtered through 0.22 μm (Millipore Durapore filters) and then ultrafiltered through a 30 kD membrane (to remove ˜11 of filtrate). Final volume 30 ml (solution A). Peptides 1, 3, 4, 5, 6 and 7 are coupled according to the same procedure.

Coupling of the Hydrophilic Amine:

24 ml of the solution obtained above (solution A) are stirred at ambient temperature and a solution 1.88 g of the polyhydroxylated amine (prepared according to the procedure described in patent EP 0 922 700 A1) is added in 5 ml of water. The pH is adjusted to 8 with a solution of HCl and 600 mg of EDCI are introduced at ambient temperature. The medium is stirred overnight, the pH is adjusted to 7.5, and the solution is filtered through a filter with a porosity of 0.22 and then ultrafiltered through a 30 kD membrane. Final volume 20 ml (solution B).

Deprotection of Lysines:

The above solution (solution B) is stirred at ambient temperature and the pH is adjusted to 10 with a solution of NaOH. After stirring for 6 h, the solution is filtered through a filter with a porosity of 0.22μ, and then ultrafiltered through a 14 kD membrane.

Coupling of Polyethylene Glycol Amines, PEG-NH₂:

The amino PEG-NH₂ 750 (O-(2-aminoethyl)-O′-methylpolyethylene glycol 750) is obtained from FLUKA®. The amino PEG 350 is prepared according to the following reaction sequence according to procedures described in the literature.

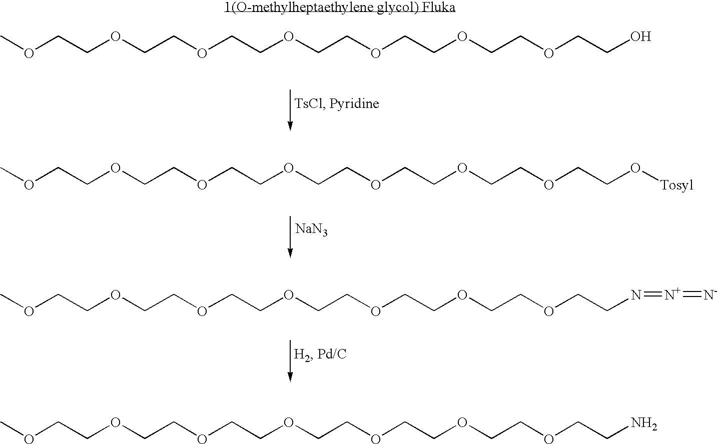

The following amino PEGs are available from the suppliers:

| | Molecular | | |
|---|---|---|---|
| Compound | weight | Trade name | Supplier |
| 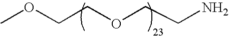 | 1088 | m-dPEG₂₄ amine | Quanta Biodesign ® |
| 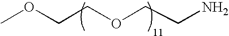 | 559 | m-dPEG₁₂ amine | Quanta Biodesign ® |
| 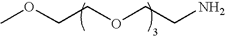 | 207 | m-dPEG₄ amine | Quanta Biodesign ® |
| 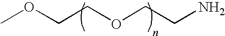 | 2000Da | mPEG-NH2 | Nektar ® |

24 ml of the USPIO-protected peptide solution (solution A) are stirred at ambient temperature and a solution of PEG 750-NH2 (1.57 g) in 5 ml of water is added. The pH is adjusted to 8 with a solution of HCl and 1 g of EDCI is added in two equal portions. The mixture is stirred overnight, the pH is adjusted to 7.5, and the solution is filtered through a 0.22μ filter and then ultrafiltered through a membrane with a cutoff threshold of 30 kD; final volume 20 ml (solution C).

The other amino PEGs are coupled according to the same procedure starting from solution A.

Characterization:

A. PEG750-USPIO-Peptide No. 2

Concentration 110 mM of iron

PCS: 26 nm

r2/r1 (20 MHz, 37° C.): 72.95/28.12=2.59

r2/r1 (60 MHz, 37° C.): 71.81/12.59=5.70

B. PEG2000-USPIO-Peptide No. 2

Concentration 20.96 mM of iron

PCS: 40 and 150 nm

r2/r1 (20 MHz, 37° C.): 273.46/36.66=7.46

r2/r1 (60 MHz, 37° C.): 312.79/16.28=19.21

C. PEG350-USPIO-peptide N°2

Concentration 142.59 mM of iron

PCS: 25 and 90 nm

r2/r1 (20 MHz, 37° C.): 101.54/33.90=2.99

r2/r1 (60 MHz, 37° C.): 104.45/15.37=6.79

### Example 2

### Coupling of the Peptides to Gadolinium Chelates

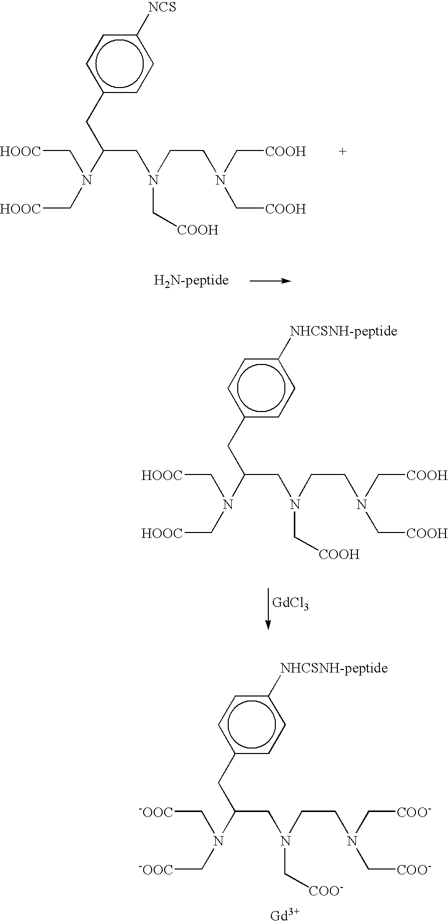

Sequence of the Coupled Peptides:

| No. | Sequence | Amount involved |
|---|---|---|
| 1 | Asp-Ala-His-Ser-Phe-SerOH | 33 mg |
| 3 | Pro-Gly-Asp-Leu-Ser-Arg-OH | 32 mg |

Condensation of the Peptides and Complexation:

The compound Bz-NCS-DTPA (Macrocyclics®) dissolved in DMF is added dropwise to a solution of peptide in 2 ml of water. The pH of the reaction mixture is adjusted to 10 with a solution of NaOH and the solution is stirred for 48 h. The pH is then adjusted to 7, and the solution is percolated through an RPC18 silica column. The fractions containing the addition compound are recovered. The ligand is then complexed with one equivalent of GdCl₃.6H₂O for 6 h.

ESI mass spectra:

Peptide No. 3-DTPAGd: [M+H]^{+:} 1184

Peptide No. 1-DTPAGd: [M+H]^{+:} 1204

Relaxivities measured at 20 MHz (37° C.):

Peptide No. 3-DTPAGd: 6.7 s⁻¹mM⁻¹

Peptide No. 1-DTPAGd: 6.8 s⁻¹mM⁻¹

Coupling of a Peptide Protected in the Form of Lateral Trifluoroacetamides:

| | | |
|---|---|---|
| 7 | 8-Amino-3,6-dioxaoctanoyl-Leu-Ile- | 350 mg |
| | Lys(tfa)-Lys(tfa)-Pro-Phe-OH | |

The peptide (350 mg, 0.323 mmol) is dissolved in 2 ml of water and the polycarboxylated compound (NCS-Bz-DTPA 420 mg, 0.646 mmol, two equivalents) in solution in water is added dropwise. The pH of the reaction mixture is adjusted to 10 with a solution of NaOH and the solution is stirred for 48 h. The pH is readjusted to 7, and the solution is percolated through an RPC18 silica column. The fractions containing the condensation product are concentrated and the ligand is complexed with one equivalent of GdCl₃.6H₂O (0.235 mmol) for 6 h.

Characterization:

Mass Spectrum of the gd Complex

Relaxivity of the Gd complex

| | r₁ to 0.47 T | r₁ to 1.4 T |
|---|---|---|
| Complexes | (s⁻¹ · mM⁻¹) | (s⁻¹ · mM⁻¹) |
| Gd-DTPA-R826 | 9.6 | 10.1 |

### Example 3

### Synthesis of a DOTA-Derived Bifunctional Chelate

### Stage 1: 5-(1,3-Dioxo-1,3-dihydroisoindol-2-yl)-2-(1,4,7,10-tetraazacyclododec-1-yl)pentanoic acid benzyl ester

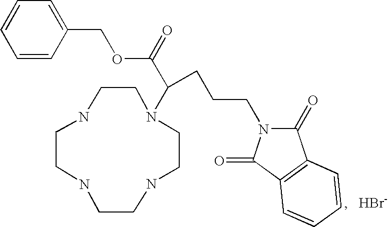

55 g of cyclen base (320 mmol) are dissolved in 550 ml of CH₃CN, to which 119.8 g of brominated derivative (2-bromo-5-(1,3-dioxo-1,3-dihydroisoindol-2-yl)pentanoic acid benzyl ester, 288 mmol) dissolved in 550 ml of CH₃CN are added dropwise. The medium is stirred at ambient temperature overnight. The precipitate is filtered off and washed thoroughly with acetonitrile. 138 g of product are obtained in the form of a white powder (corrected yield 81.3%).

TLC: CH₂Cl₂/MeOH/NH₄OH at 25% (80/40/3)

Revelation UV and CuSO₄

Rf: 0.3

### Stage 2: 5-(1,3-Dioxo-1,3-dihydroisoindol-2-yl)-2-(4,7,10-tris(ethoxycarbonylmethyl)-1,4,7,10-tetraazacyclododec-1-yl)pentanoic acid benzyl ester

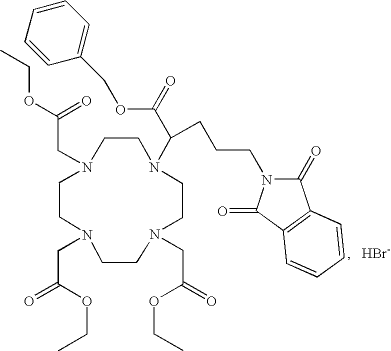

60 g of the compound obtained in stage 1 (102 mmol) and 50.1 g of Na₂CO₃ (464 mmol) are added to a solution of 59.1 g of ethyl bromoacetate (Aldrich®, 358 mmol) in CH₃CN (1.11). The reaction medium is heated at 80° C. under a covering of argon overnight. After removal of the precipitate, the filtrate is concentrated and washed thoroughly with CH₃CN. The product is crystallized from CH₃CN by dropwise addition of Et₂O. 89.8 g of product are obtained in the form of a white solid (corrected yield 100%).

TLC: CH₂Cl₂/MeOH (9/1)

Revelation UV and KMnO₄; Rf: 0.4

### Stage 3: 5-Amino-2-(4,7,10-tris(carboxymethyl)-1,4,7,10-tetraazacyclododec-1-yl)pentanoic acid

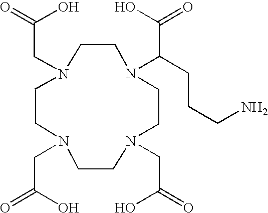

In a 5 liter reactor, a solution of 54 g of compound obtained in stage 2 (64 mmol) in 37% hydrochloric acid (1.8 1) is refluxed overnight. After cooling and filtration, the filtrate is concentrated and purified on silanized silica (elution with water). After evaporation under reduced pressure, the product is washed with ether. 45 g of product are obtained in the form of a white solid. The product is desalified by passing it over Off resin. 30 g of product are isolated in the form of white crystals (yield 100%).

HPLC: Hypercarb® 5μ, 200X4.6, 250 Å; Solvent A: 0.037 N sulfuric acid

Solvent B: CH₃CN; UV detection at 201 nm; Tr: 18 min.

### Stage 4: 5-Amino-2-(4,7,10-tris(carboxymethyl)-1,4,7,10-tetraazacyclododec-1-yl)pentanoic acid gadolinium complex

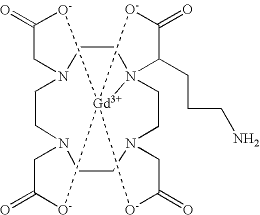

7.2 g of the compound obtained in stage 3 (16 mmol) are dissolved in 70 ml of water and the pH is adjusted to 5.5 by adding 6N hydrochloric acid. 2.9 g of Gd₂O₃ (8 mmol) are added and the reaction medium is heated at 80° C. The pH of the solution steadily increases and should be maintained between 5.2 and 5.7 by adding 6N hydrochloric acid dropwise. After two hours, the pH stabilizes at 5.7. The slight cloudiness is filtered out through a Whatman® filter and the filtrate is concentrated. 11.1 g of product are obtained in the form of white flakes (corrected yield 100%).

HPLC: Hypercarb® 5μ, 200X4.6, 250 Å; Solvent A: 0.037 N sulfuric acid

Solvent B: CH₃CN; UV detection at 201 nm; Tr: 10 min.

### Stage 5: 5-(2-Ethoxy-3,4-dioxocyclobut-1-enylamino)-2-(4,7,10-tris(carboxymethyl)-1,4,7,10-tetraazacyclododec-1-yl)pentanoic acid gadolinium complex

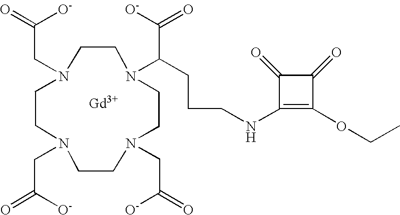

8 g of compound obtained in stage 4 are dried by azeotropic distillation with toluene, and then suspended in 90 ml anhydrous DMSO under a covering of argon. 2.8 ml of screen-dried Et₃N (1.7 eq.) and 5 g of diethyl squarate (Aldrich®, 2.5 eq.) are then added. The medium is stirred at ambient temperature under a covering of argon for 1 hour. The mixture is precipitated from 600 ml of ether. The solid obtained is filtered off, and then washed with dichloromethane. After filtration and drying, 7.5 g of a white solid (yield of 81.5%) are recovered.

HPLC: Symmetry C18, 5μ, 250X4.6, 100 Å

A: water with TFA, pH=2.7; B: CH₃CN; Detection at 201 and 254 nm; Tr: 19.8 min

### Example 4

### Peptide Couplings

| No. | Sequence | MW |
|---|---|---|
| 1 | Asp(tBu)-Ala-His(trt)-Ser(tBu)-Phe- | 1073.31 |
| | Ser(tBu)OH | |
| 2 | Leu-Ile-Lys(Boc)-Lys(Boc)-Pro-Phe-OH | 945.22 |
| 3 | Pro-Gly-Asp-(tBu)-Leu-Ser(tBu)- | 1008.25 |
| | Arg(Pbf)-OH | |
| 4 | Gly-Asp(tBu)-Ala-His(trt)-Ser(tBu)-Phe- | 1130.36 |
| | Ser(tBu)OH | |
| 5 | γ-Abu-Asp(tBu)-Ala-His(trt)-Ser(tBu)- | 1158.42 |
| | Phe-Ser(tBu)OH | |
| 6 | 8-Amino-3,6-dioxaoctanoyl-Asp(tBu)-Ala- | 1218.47 |
| | His(trt)-Ser(tBu)-Phe-Ser(tBu)OH | |
| 7 | 8-Amino-3,6-dioxaoctanoyl-Leu-Ile- | 1082.16 |
| | Lys(tfa)-Lys(tfa)-Pro-Phe-OH | |

Coupling of Protected Peptides:

Stage 1: Coupling of Peptides Nos. 1, 2, 3, 4, 5, 6 or the Squarate Derivative

The compound obtained in stage 5 of example 3 (100 mg, 1.35×10⁻⁴ mol) is dissolved in 15 ml of an aqueous solution of Na₂CO₃, pH 9.4. The protected peptide (1.6×10⁻⁴ mol) is introduced while maintaining the pH at 9.4 by adding Na₂CO₃. If the peptide is not soluble in water, a few drops of DMF are added until complete dissolution is obtained. After reaction at ambient temperature for 48 h, the medium is precipitated from an ethanol/diethyl ether mixture. The precipitate is filtered off and then dried.

Stage 2: Deprotection

The compound obtained in stage 1 above is dissolved in a mixture of 10 cm³ of TFA/TIS/H₂O in proportions of 90/5/5. The medium is stirred for 5 h at ambient temperature and the solvent is then evaporated off under reduced pressure. The residue is taken up in ethyl ether and the precipitate is filtered off and then dried. The product is then purified by preparative HPLC on a Symmetry® column with an eluent constituted of water/TFA, pH 3/CH₃CN.

Coupling of a TFA-Protected Peptide, Peptide No. 7:

210 mg of the compound obtained in stage 5 of example 3 are dissolved in 20 ml of water and the pH is adjusted to 9 with a solution of Na₂CO₃. 350 mg of protected peptide (peptide No. 7) are added and the pH is adjusted to 9.2. The medium is stirred at ambient temperature and the solution is then dialyzed through a membrane with a cutoff threshold of 1 kD for 48 h and then chromatographed on an RP-18 column (MeOH/water mixture (50/50)).

| | | | Mass |
|---|---|---|---|
| No. | Structure | MW | spectrometry |
| 1 | 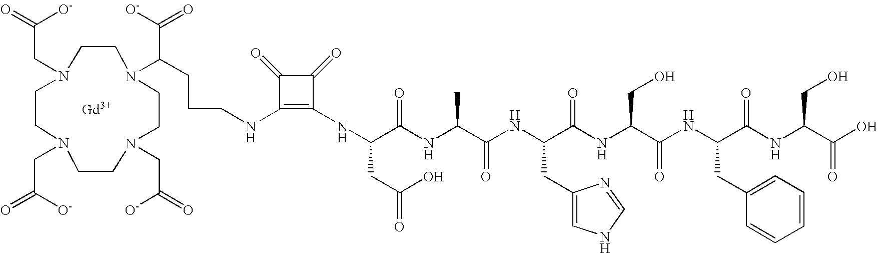 | 1355.43 | ES-m/z: 1354.2 |
| 2 | 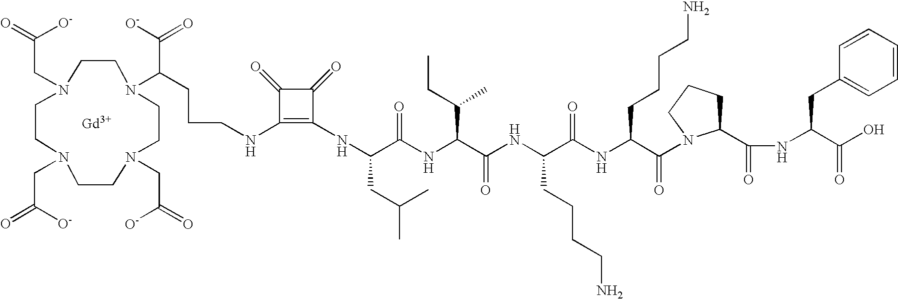 | 1437.75 | ES-m/z: 1436.3 |
| 3 | 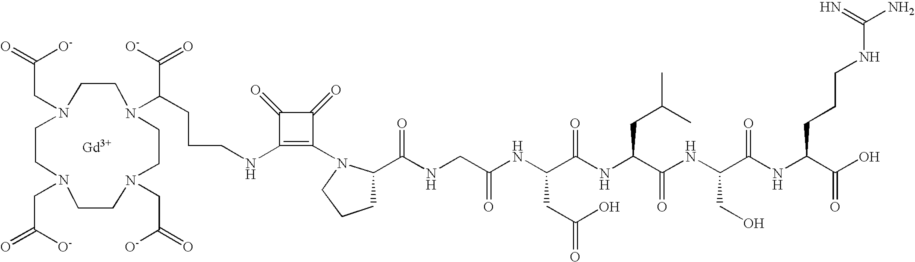 | 1336.47 | ES-m/z: 1335.1 |
| 4 | 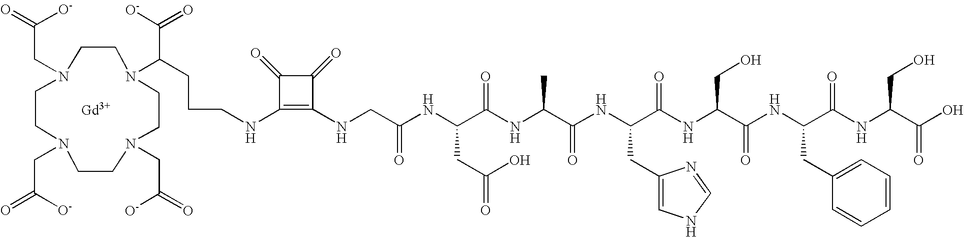 | 1412.48 | ES-m/z: 1412 |
| 5 | 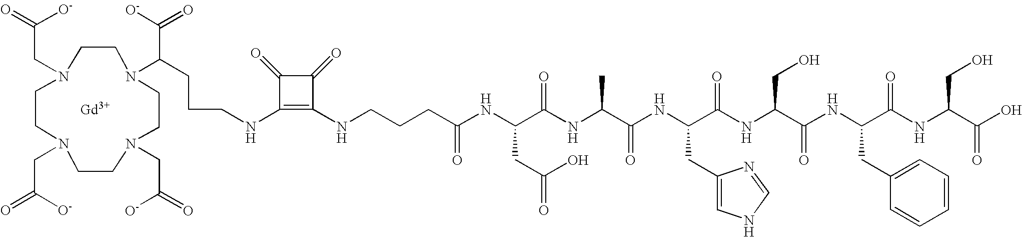 | 1440.53 | ES-m/z; 1439 |
| 6 | 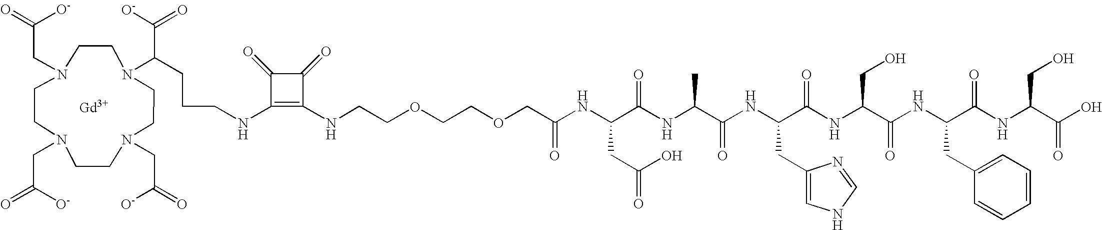 | 1500.59 | ES-m/z: 1448 |
| 7 | 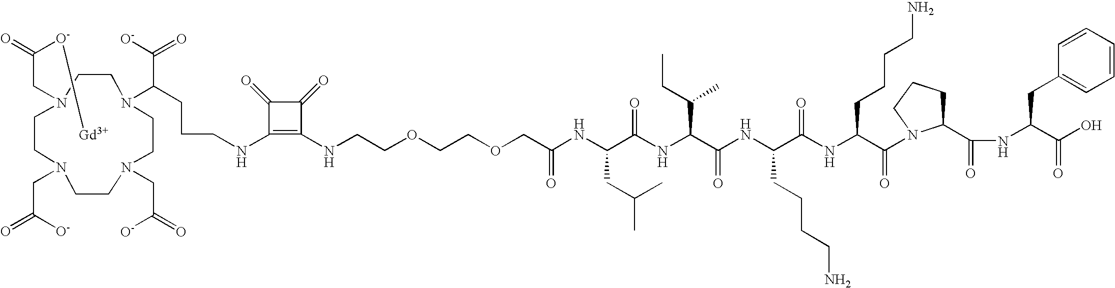 | 1582.91 | ES-m/z: 1582 |

### Example 5

### Couplings of DOTA- and PCTA-Derived Chelates

DOTA Carboxylate:

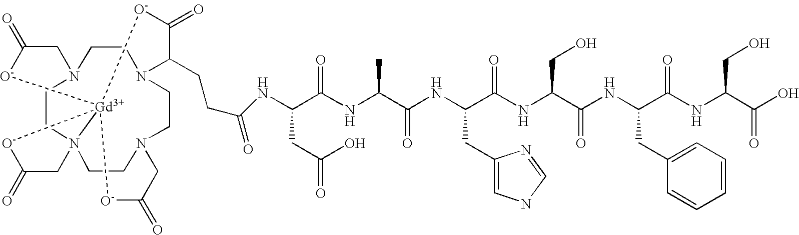

Peptide coupling of a DOTA carboxylate to a protected peptide in the presence of EDCI and deprotection in TFA.

MS: ES-, M/Z=1274 with z=1

DOTA Thiourea:

Condensation in a basic medium with an isothiocyanate according to example 2 and deprotection in TFA.

| | | | Mass |
|---|---|---|---|
| No. | Structure | MW | spectrometry |
| 1 | 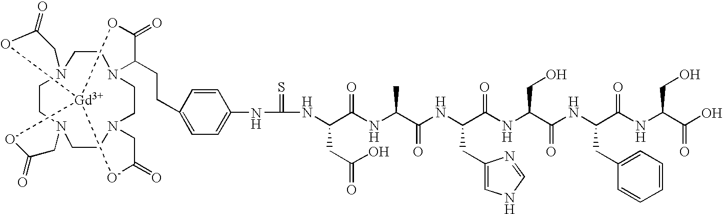 | 1381.53 | ES-m/z: 1379.5 |
| 3 | 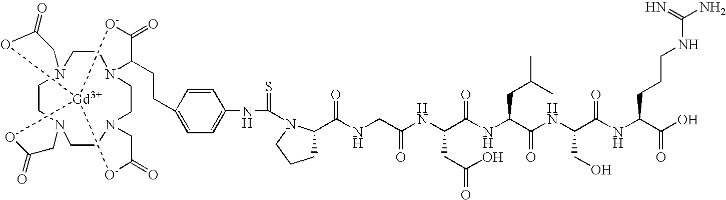 | 1362.57 | ES-m/z: 1361 |

PCTA Carboxylate:

Peptide coupling and deprotection in TFA.

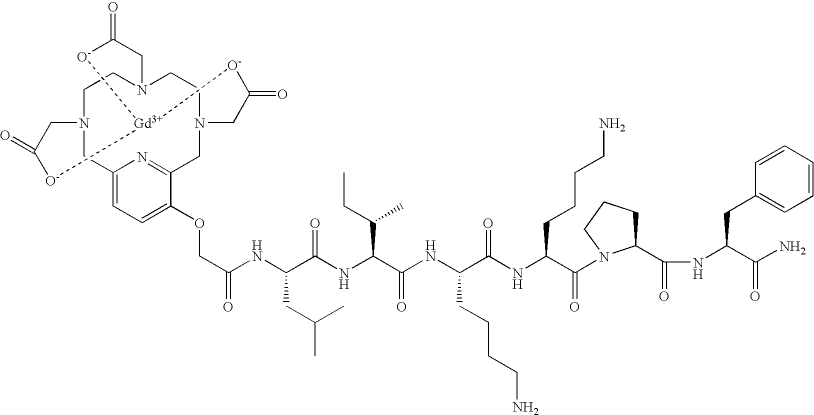

MS: ES+, M/Z=1336 with z=1

PCTA Thiourea:

Condensation in a basic medium with an isothiocyanate according to example 2 and deprotection in TFA.

| | | | Mass |
|---|---|---|---|
| No. | Structure | MW | spectrometry |
| 1 | 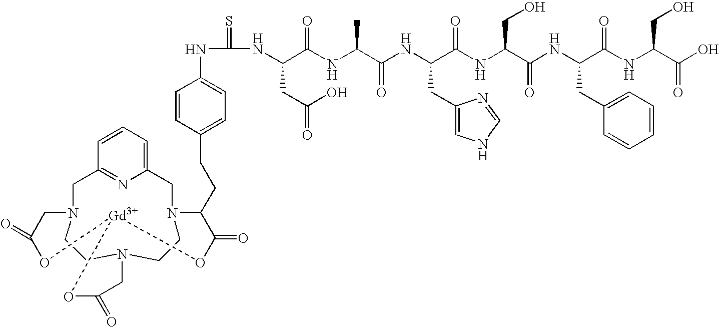 | 1358.52 | ES + m/z: 1359.8 |
| | Molecular Weight = 1358.52 | | |
| 3 | 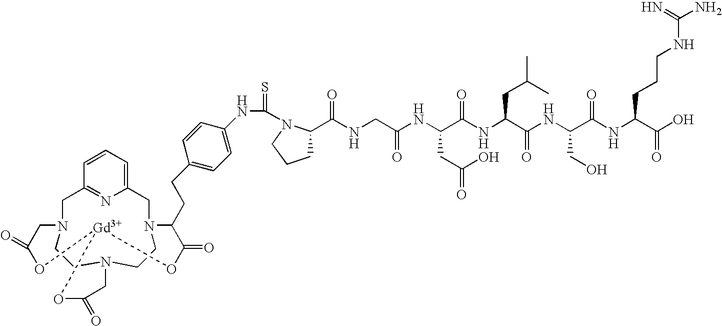 | 1339.56 | ES + m/z: 1341 |

PCTA Squarate:

According to example 4, stages 1 and 2.

| | | | Mass |
|---|---|---|---|
| No. | Structure | MW | spectrometry |
| 1 | 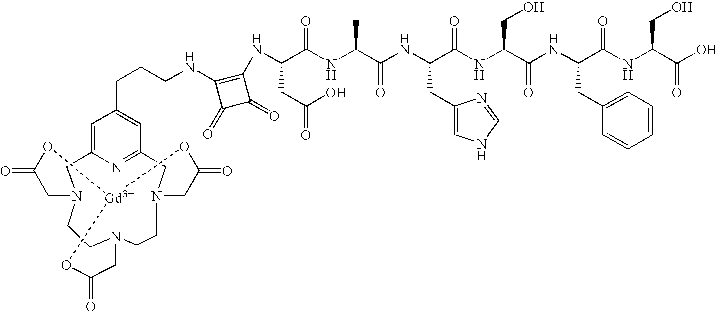 | 1332.42 | ES + m/z; 1333.6 |
| 2 | 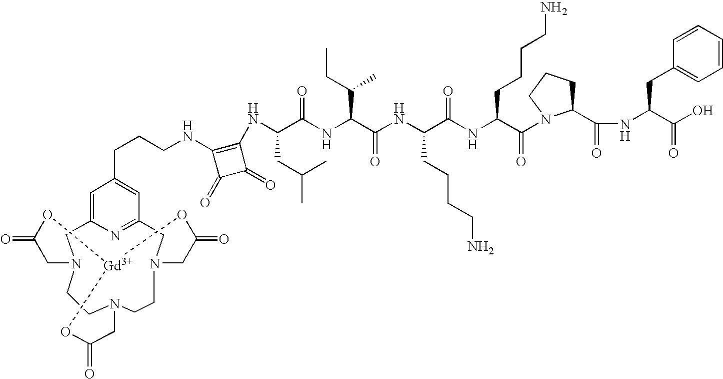 | 1414.74 | ES + m/z: 1416 |
| 3 | 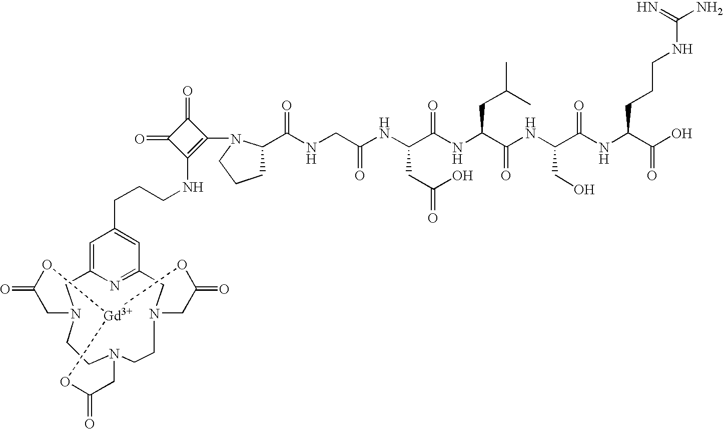 | 1313.46 | ES + m/z: 1314 |

### Example 6

### Preparation of Lipopeptides

Stage 1: Pentafluorophenyl Hexadecanoate

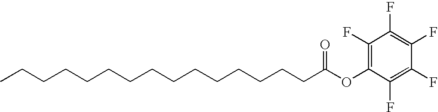

3.1 g of dicyclohexylcarbodiimide (DCC) in 20 ml of dioxane are added, at 0° C., to a solution constituted of 3.85 g of hexadecanoic acid and 2.76 g of pentafluorophenol in 20 ml of dioxane and 8 ml of DMF. The mixture is stirred overnight at ambient temperature and the reaction medium is then filtered and the solution obtained is evaporated under reduced pressure. The oil thus recovered is taken up in cyclohexane, so as to give a white solid (5 g).

Melting point: 42-44° C.

Stage 2: Peptide Acylation

The peptides are solid-phase synthesized using 2.5 equivalents of each Fmoc-protected amino acid at each coupling cycle. The carboxylic acids are activated with HATU, N-methylmorpholine in DMF. The Fmoc groups are cleaved by treatment with piperidine (20% in DMF). After introduction of the final amino acid of the peptide sequence and cleavage of the Fmoc protective group, the N-terminal amine of the peptide is acylated using the compound prepared in stage 1 (2.5 equivalents) dissolved in CH₂Cl₂, in the presence of HoBt and of N-methylmorpholine. The peptide is then released from the resin and the groups protecting the side functions are cleaved through the action of a trifluoroacetic acid/thioanisole mixture (95/5) for 30 minutes at 0° C. and then 2 h at ambient temperature. The resin is removed and the solvent is evaporated off under reduced pressure. The lipopeptide is precipitated from ethyl ether. The products are purified by preparative HPLC on a Vydac ODS® column, elution being carried out with a water/acetonitrile/TFA mixture.

| | Molecular | ES + |
|---|---|---|
| Structure | weight | m/z |
| 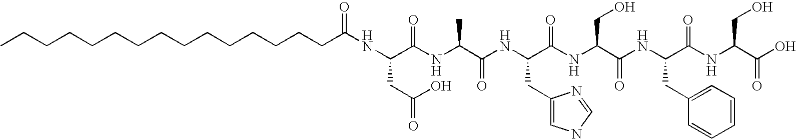 | 901.08 | 902.5 |
| 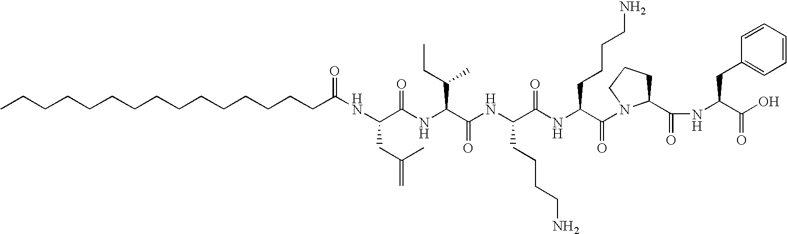 | 983.40 | 986 |
| 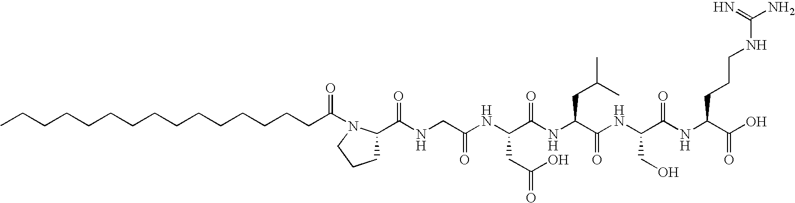 | 882.12 | 884 |

### Examples Part II

### Demonstration of the Effectiveness of PEPTIDES P

### Example II.1

### Peptide LIKKPF (SEQ ID No 11)

The affinity of the peptide LIKKPF (SEQ ID No 11) is measured on immobilized PS (ELISA plate). The peptide is incubated at increasing concentrations of 5×10⁻¹⁴ M to 4.2×10⁻⁷ M. Annexin V-biotin is then added at the concentration of 5×10⁻¹⁰ M, which corresponds to its K_{d} value for PS. Incubation at 37° C. is sustained for 1.5 hours. The bound annexin V is detected by adding a solution of streptavidin-HRP, and then a solution of ABTS substrate containing 0.05% of H₂O₂. The OD₄₀₅ is measured in order to calculate the IC₅₀ value of the peptide.

### Example II.2

### USPIO-LIKKPF Contrast Product

The affinity of the USPIO-LIKKPF is measured on immobilized PS (120 μg/ml). After saturation of the nonspecific sites, the USPIO-LIKKPF is incubated at increasing concentrations (122 nM-4 mM with respect to iron) for 2 hours at 37° C. After washing of the wells, the molecules bound to the PS are digested in one volume of 5N HCl. The samples are digested in one volume of 5N HCl, for 48 h at 37° C., and the iron is assayed using Prussian blue (OD₆₃₀).

The in vitro MRI is carried out on apoptotic (or control) cells preincubated with the USPIO-LIKKPF or unfunctionalized USPIO (3 or 4 mM with respect to iron) for 2 hours at 37° C. Jurkat cell apoptosis is induced by a treatment with 2 μM camptothecin for 24 hours. After incubation with the USPIO, the labeled cells are transferred into a 0.2% gelatin solution for analysis by MRI (Bruker Avance-200, 4.7 T, TR/TE=3000/20 ms, 30 echos, matrix=256×256, FOV=4 cm, slice thickness=1 mm) in ELISA plate wells.

### Example 11.3

### Gd-DTPA-LIKKPF Contrast Product

The peptides LIICKPF (SEQ ID No 11) and FKIPKL (“scrambled” peptide—mixed sequence) are grafted separately to a Gd-DTPA chelate. The peptides protected with TFA on two Lys are attached to the 8-amino-3,6-dioxooctanoyl linker. The protected peptide is coupled to DTPA-isothiocyanate (NCS-Bz-DTPA), and then the Lys are deprotected in a basic medium.

Apoptotic Jurkat cells in suspension (2×10⁶ cells/nil) are incubated in the presence of the Gd-DTPA-LIKKPF product at the concentrations of 400, 200 and 100 μM with respect to Gd. The cells are incubated for 2 hours at ambient temperature. After rinsing and centrifugation, the cell pellet is mineralized under acidic conditions and the amounts of Gd are measured by ICP-MS (inductively coupled plasma-mass spectrometry).

The targeting effectiveness of the Gd-DTPA-LIKKPF product by MRI was also evaluated on a model of apoptotic liver in the mouse. The apoptosis is induced by means of an i.v. injection (caudal vein) of anti-Fas monoclonal antibody in anesthetized Balb/c mice (Blankenberg et al., 1998, 1999; Rodriguez et al., 1996).

Before the MRI acquisition, the mice are anesthetized with pentobarbital. The experiments are carried out using a 200 MHz Bruker spectrometer (4.7 T) equipped with a vertical magnet and with a mini-imaging system. The images are acquired using an MSME (multi-slice-multi-echo) sequence with the following parameters: TR/TE=307.4/14.7 ms, matrix=256, FOV=5 cm, slice thickness=3 mm, eight axial slices (which completely cover the abdominal region including the kidneys), TA=5 minutes 14 seconds. The images are acquired before and after injection of the contrast products, up to 1.5 hours. The signal intensity (SI) is measured in the liver. The SI value is also measured in a tube filled with a 2% aqueous solution of gelatin enriched with 50 μM Gd-DTPA, used as a reference product, in a region outside the image of the mouse which corresponds to the background noise. The MRI protocol is the following: (1) the precontrast MRI begins 1 h15 after the injection of anti-Fas; (2) the contrast product is injected into the femoral vein at the dose of 60 μmol Gd/kg approximately 1 h45 after the injection of anti-Fas; (3) several post-contrast acquisitions are carried out for 1 h30. A competition protocol is carried out by injection of 100 μmol peptide/kg, 10 minutes before the injection of the Gd-DTPA-LIKKPF contrast product.

The targeting effectiveness of the Gd-DTPA-LIKKPF product was also evaluated on another model of apoptosis: the apoE^{-/-} mouse, which is a model of atherosclerosis.

Female C57B1 ApoE^{-/-} mice approximately 15 months old are subjected to a cholesterol-rich diet for 3 months before the MRI studies. For the acquisition by MRI, the animals are anesthetized with pentobarbital. The Gd-DTPA-LIKKPF contrast agent is injected i.v. at the dose of 100 μmol Gd/kg. All the images are acquired at the level of the abdominal aorta, in particular the region close to the kidney, which is known for the development of atheroma plaques owing to the presence of the arterial branches.

The parameters of the RARE (Rapid Acquisition with Relaxation Enhancement) sequence are adjusted using a reference tube filled with a 1 mM solution of Gd-DTPA. The image acquisition parameters are the following: TR=470.9-1048.5 ms, TE=4 ms, RARE factor=1-4, NEX=4, matrix=256×256, FOV=2.3 cm, spectral width=33.33 kHz, slice thickness=0.8 mm, spatial resolution=90 μm.

For the two models, the SI values are measured in various regions of interest (at the level of the arterial wall of the abdominal aorta or whole liver) using the OSIRIS image analysis software. The regions are first drawn on the post-contrast images, and then duplicated on the pre-contrast images. The SI value is measured on all the image slices where the arterial wall and the liver are visible. Finally, the SI values obtained for serial slices of aorta over a length of 3.2-8 mm are averaged for each animal. The % ΔSNR is calculated according to the following equation:

% ΔSNR=[(SIₚₒₛₜ/SDₙₒᵢₛₑ)−(SIₚᵣₑ/SDₙₒᵢₛₑ)]/[SIₚᵣₑ/SDₙₒᵢₛₑ]×100 where SDₙₒᵢₛₑ is the standard deviation of the noise measured on a region outside the animal.

II.4 Results: Affinity of the PEPTIDES P

The applicant identified several consensus sequences:
VLGERG (SEQ ID No 5) and LIKKPF (SEQ ID No 11);
D-A-H-S-X7-S (SEQ ID No 2), where X7 may be F or L;
P-G-D-L-X8-X9 (SEQ ID No 3), where advantageously X8 is chosen from S or V and X9 is chosen from T or R;
H-G-X10-L-S-X11 (SEQ ID No 4) and functional equivalents thereof, where advantageously X10 is chosen from D or H, and X11 is chosen from T or I.

The affinity (in order of magnitude) of these peptides for PS (apparent dissociation constant K*_{d}) is between 10⁻⁶ and 10⁻⁹M.

The high-specificity peptide LIKKPF (SEQ ID No 11) was particularly well characterized. The specificity of interaction of the peptide with PS is confirmed by means of the annexin V competition assay. The IC₅₀ value of annexin V is equal to 1.08×10⁻⁹ M (FIG. 1).

### Example 11.5

### Peptide LIKKPF (SEQ ID No 11)

The in vitro assay on immobilized PS makes it possible to calculate an IC₅₀ value (FIG. 2).

### Example 11.6

### USPIO-PEG-PEPTIDE P Contrast Product with PEPTIDE P Being LIKKPF (SEQ ID No 11) (Also Denoted USPIO-LIKKPF)

The assay for interaction of the USPIO-LIKKPF with the immobilized PS makes it possible to measure a K*_{d} value of 7.85×10⁻⁸ M. The specificity of interaction of the USPIO-LIKKPF with the PS is verified by means of an annexin V competition assay.

The IC₅₀ value is equal to 4.2×10⁻⁷ M (FIG. 3).

The MRI on cells shows that the USPIO-LIKKPF binds specifically to the apoptotic cells. This is because the MRI signal intensity recorded with these cells is weaker than that obtained with the control cells. Furthermore, the decrease in MRI signal induced by incubation with the USPIO-LIKKPF is greater than that recorded with the unfunctionalized USPIO (USPIO-PEG not coupled to the peptide LIKKPF (SEQ ID No 11))-cf. FIG. 4 (apoptotic cells; the left-hand column corresponds to the 3 mM concentration, the right-hand column corresponds to the 4 mM concentration of Fe).

All these results demonstrate the specific targeting of the USPIO-LIKKPF for apoptotic cells.

### Example II.7

### Gd-DTPA-LIKKPF Contrast Product

The grafting of the peptide LIKKPF (SEQ ID No 11) to the Gd-DTPA contrast product makes it possible to conserve the targeting of apoptotic cells. Specifically, the ICP-MS assaying of apoptotic cells incubated with Gd-DTPA-LIKKPF proves that their interaction, which is concentration-dependent, is greater than the uptake of the product by the control cells (FIG. 5).

After i.v. injection of 60 μmol Gd/kg of product, the dynamic monitoring of the MRI signal of the mouse apoptotic liver (FIG. 6) shows an enhancement with the Gd-DTPA-LIKKPF which is greater than that measured with the Gd-DTPA-FKIPKL (scrambled) or the unfunctionalized Gd-DTPA. Preinjection of the peptide LIKKPF (SEQ ID No 11), at the dose of 100 μmol/kg, blocks the enhancement caused by the Gd-DTPA-LIKKPF, thereby attesting to the specificity of interaction between the Gd-DTPA-LIKKPF and PS. Moreover, the three products (Gd-DTPA-LIKKPF, Gd-DTPA-FKIPKL and Gd-DTPA) enabled only a partial signal enhancement to be recorded in the healthy mouse. All these data prove that the Gd-DTPA-LIKKPF contrast product specifically targets apoptosis in vivo via PS.

### Example II.8

### Imaging of Apoptotic Vascular Tissue

In a model of atherosclerosis in the mouse (apoE^{-/-} mouse), the i.v. injection of Gd-DTPA-LIKKPF product, at the dose of 100 gmol Gd/kg, makes it possible to dynamically detect a signal enhancement greater than that measured with the nongrafted product (Gd-DTPA), at the level of the abdominal aorta (FIGS. 7 and 8—RARE sequence, 27 minutes after injection of contrast product—MRI of serial slices of abdominal aorta over a length of 3.2 mm). This result indicates that the Gd-DTPA-LIKKPF product is capable of specifically targeting the atheroma plaque, via interaction with PS.

### Example II.9

### Imaging of Apoptotic Nervous Tissues

In a model of Parkinson's disease, with the injection of USPIO-LIKKPF product (product of example 1)

MRI studies were carried out on C57BI/6 mice treated with MPTP (or its metabolite MPP+ for cell cultures), a neurotoxin which induces a “parkinsonien” syndrome.

In cell culture, the death of dopaminergic neurons by apoptosis was verified using rat brains, by immunohistochemistry and detection of caspase 3 enzyme activity. For the MRI detection in cell culture, the healthy cells and cells treated with MPP+ were brought into contact with USPIO-LIKKPF. The diseased cells show a darker signal than the others, demonstrating the targeting of the peptide to the apoptotic cells. The standardized relaxation rate and the iron concentration show that the diseased cells have taken up a larger amount of vectorized contrast agent.

The immunohistochemistry and the MRI are very revealing. The diseased and healthy mice were studied on the 4th, 5th and 6th day of treatment, and one and three weeks after the end of the treatment with MPTP. The imaging shows a change in the signal over the course of the treatment, the targeting being more marked at the 5th day, and then decreasing thereafter. The slices prepared with these nonvectorized USPIO (without peptide) do not show a marked targeting. The MRI localization of the diseased regions corresponds to the regions containing dopaminergic neurons (immunohistochemistry: detection of dopaminergic neurons by measuring tyrosine hydroxylase in the central gray nuclei).

1. A compound of general formula (I) below:
Signal-Linker-Peptide (I)
in which:
Signal represents a signal entity chosen from:
a chelate which may or may not be coupled to a paramagnetic metal M or to a radionucleide,
a superparamagnetic nanoparticle coated with an organic layer, and p2 a label for optical imaging;
Linker, which may or may not be present, represents a chemical bond, and
Peptide represents a peptide comprising an apoptosis-targeting peptide, the apoptosis-targeting peptide being chosen from the peptides having the formula below:

| | |
|---|---|
| X1-X2-X3-X4-X5-X6 (1) | (SEQ ID No 1) |

in which X1 and X2 represent, independently of one another, leucine or isoleucine, X3 and X4 represent lysine, X5 represents proline and X6 represents phenylalanine;

| | |
|---|---|
| D-A-H-S-X7-S (2) | (SEQ ID No 2) |

in which X7 represents phenylalanine or leucine;

| | |
|---|---|
| P-G-D-L-X8-X9 (3) | (SEQ ID No 3) |

in which X8 represents serine or valine and X9 represents threonine or arginine;

| | |
|---|---|
| H-G-X10-L-S-X11 (4) | (SEQ ID No 4) |

in which X10 represents aspartic acid or histidine, and X11 represents threonine or isoleucine;

| | |
|---|---|
| V-L-G-E-R-G (5); | (SEQ ID No 5) |

and the pharmaceutically acceptable salts of these compounds, with the exception of the compounds of the formulae below:

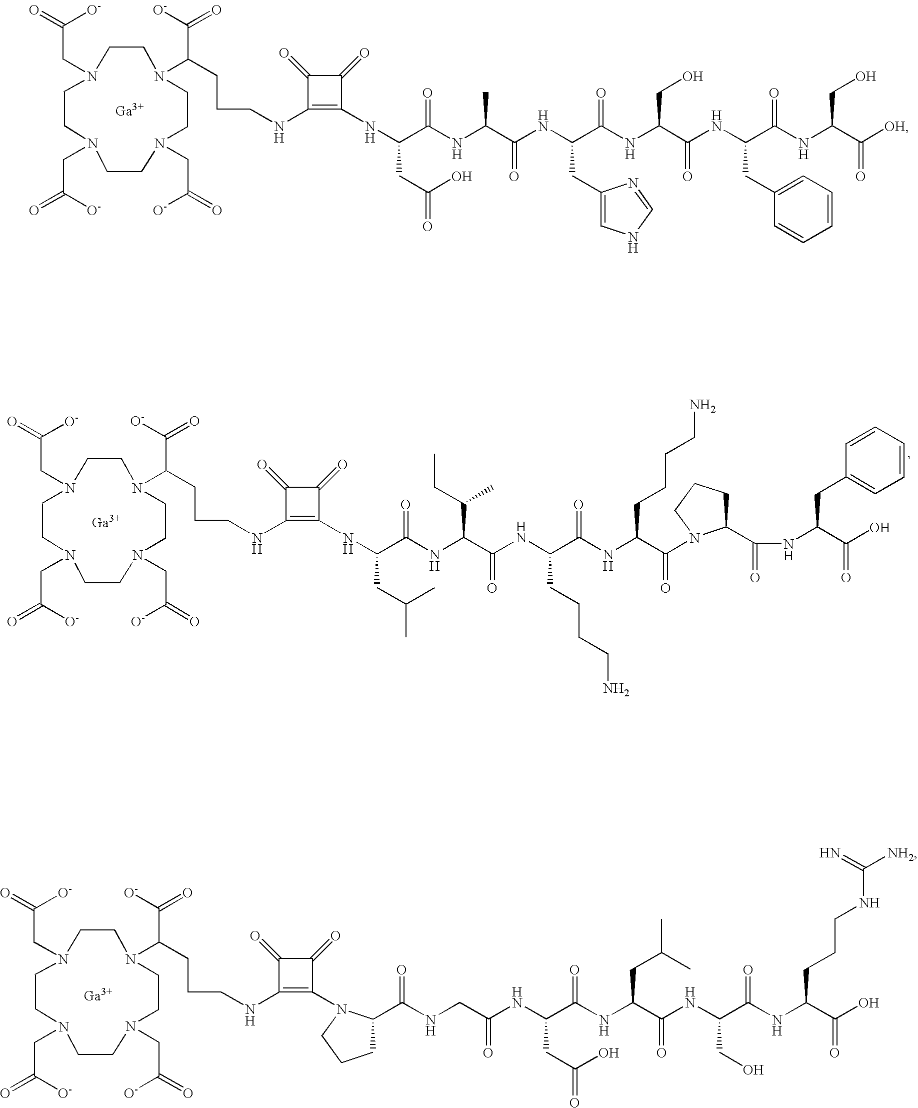

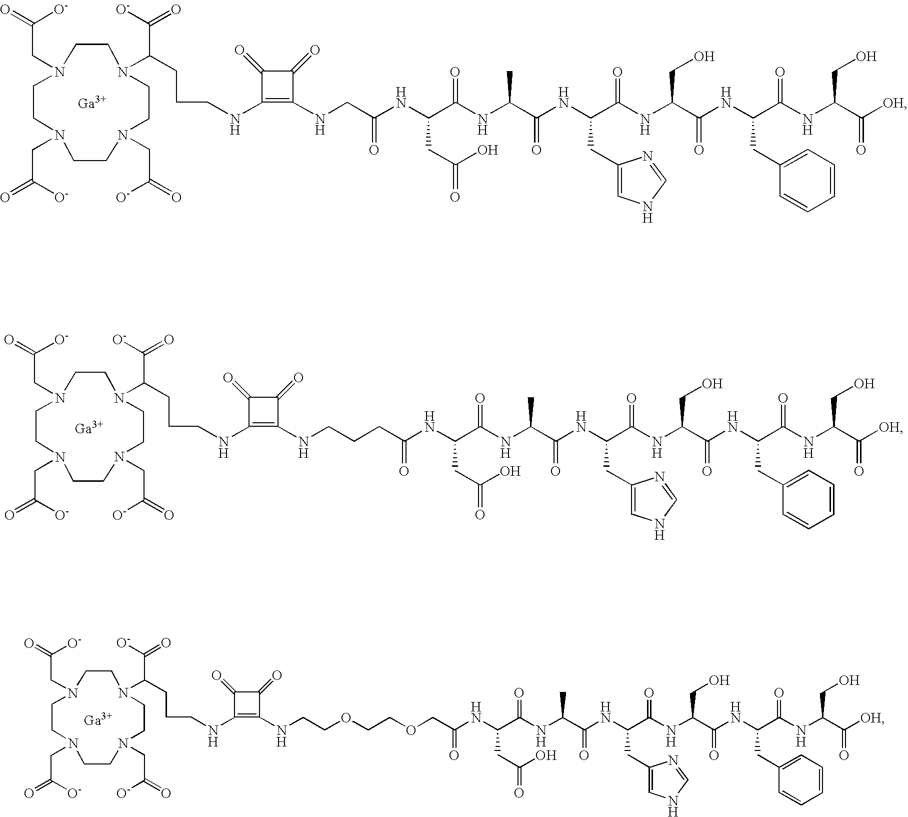

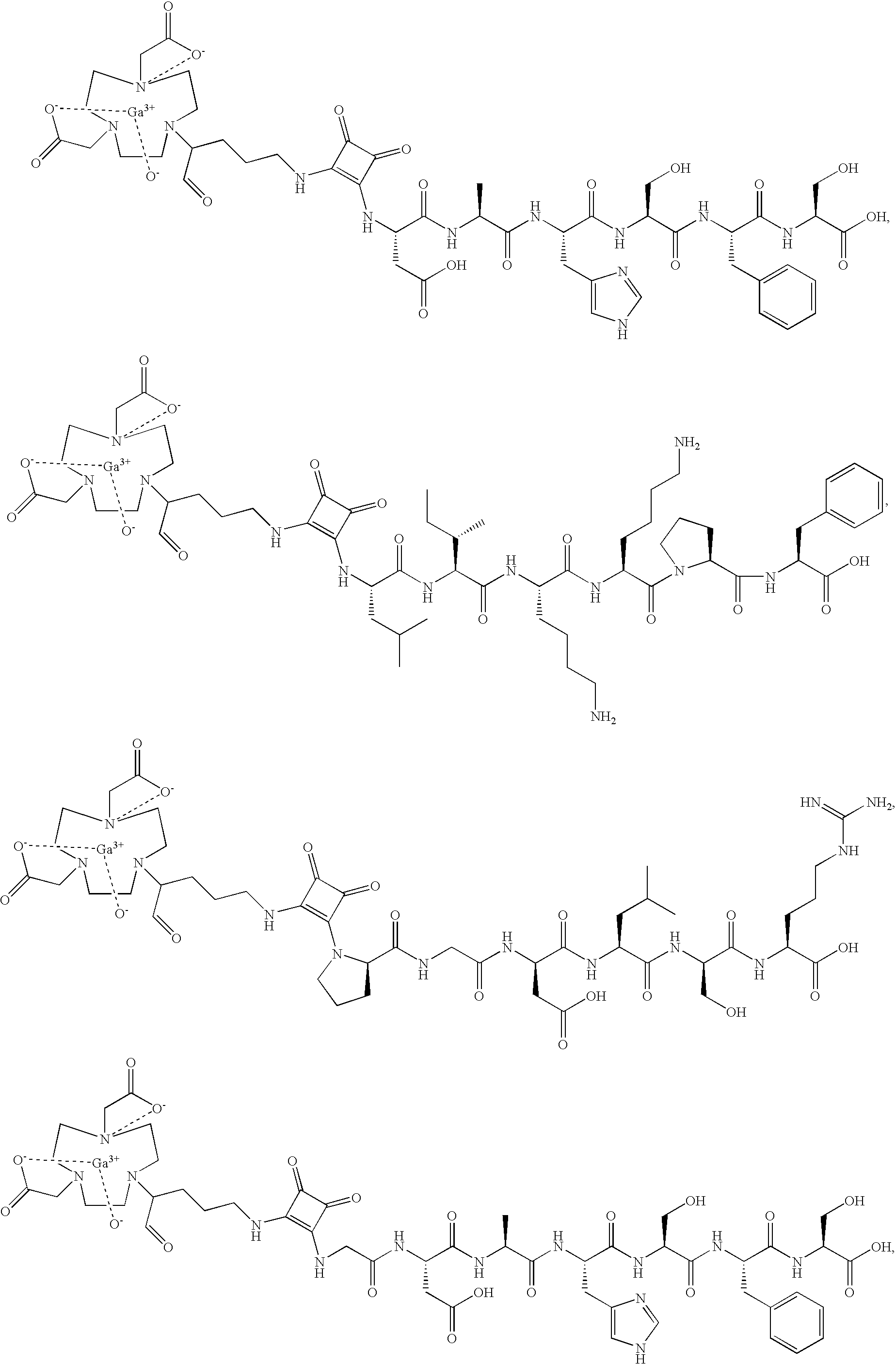

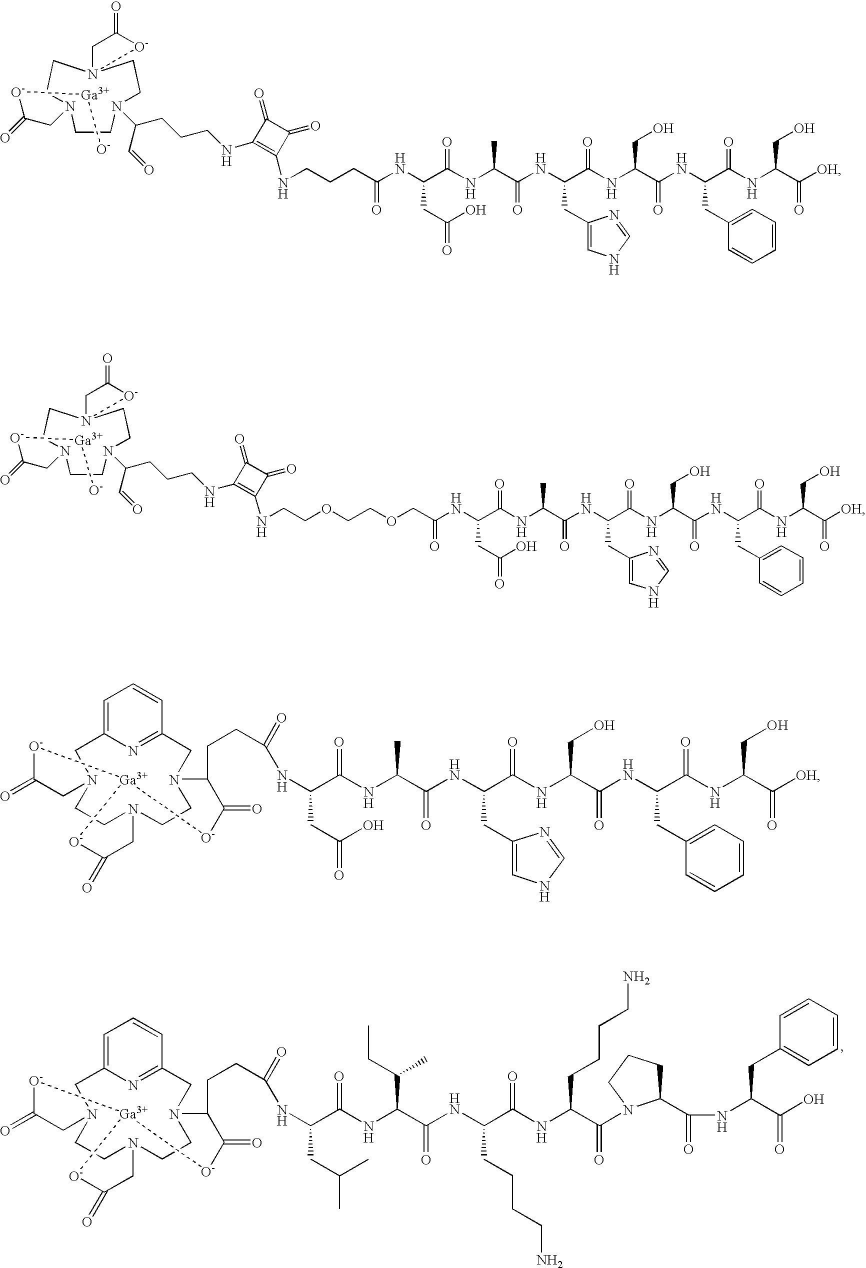

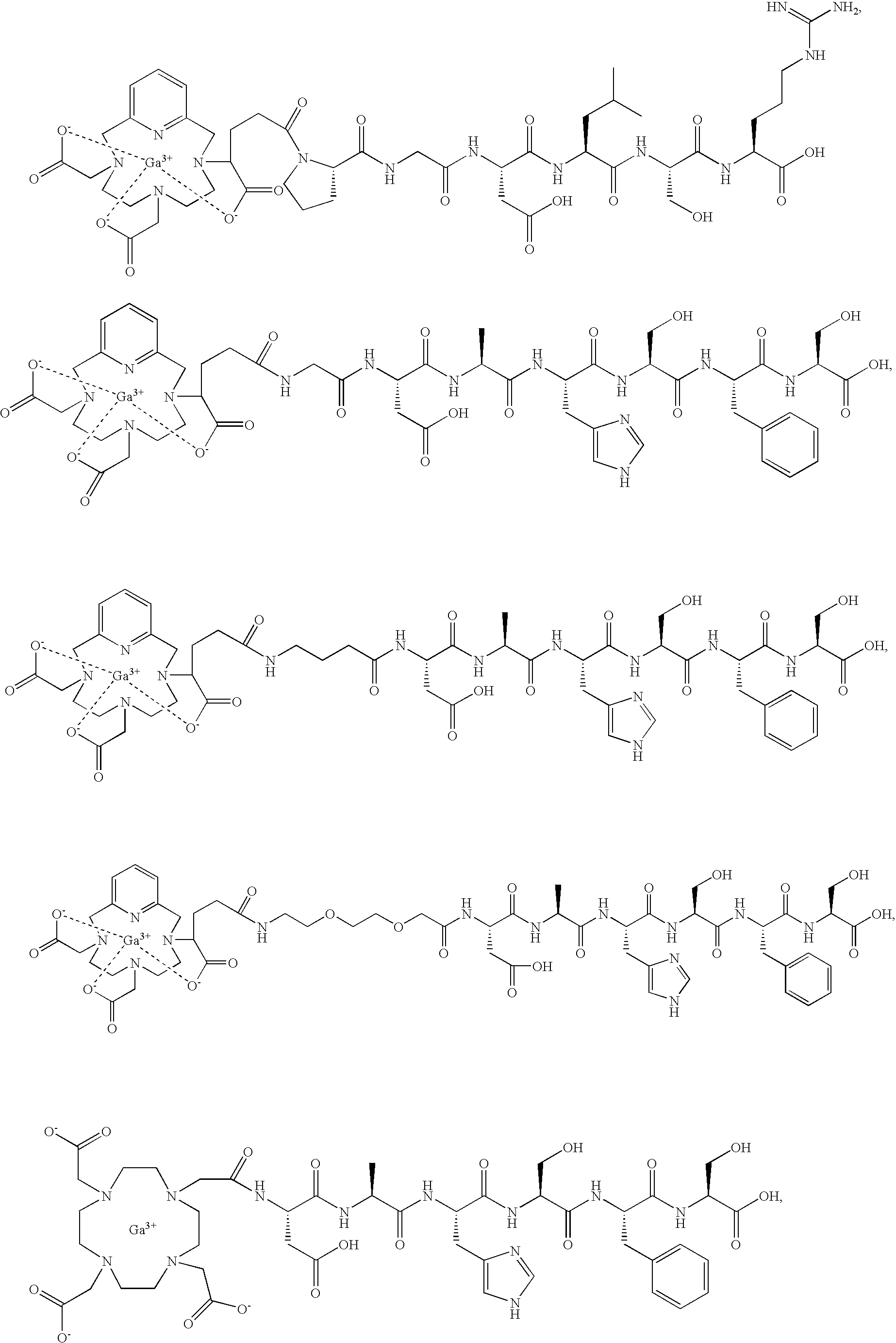

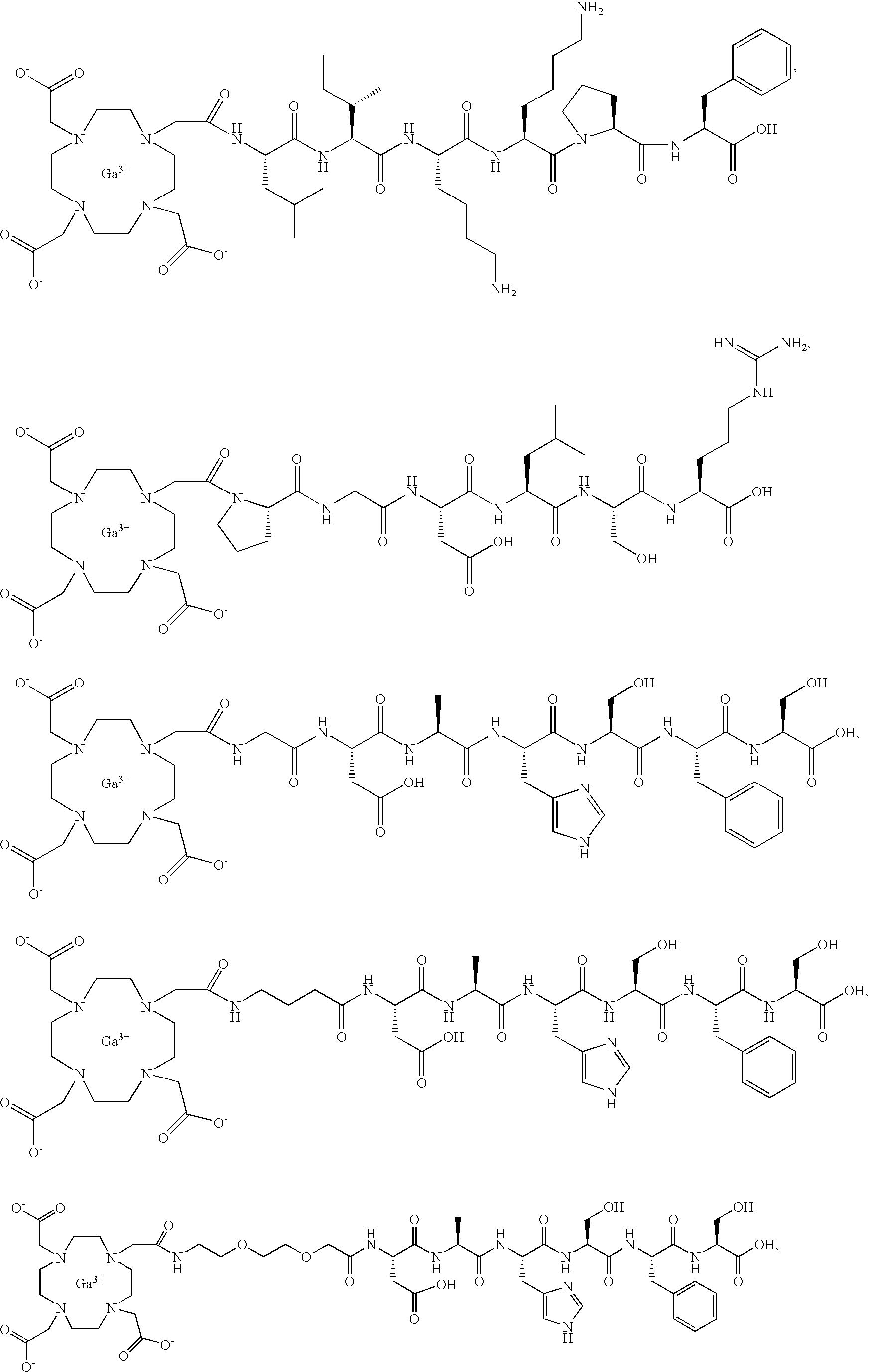

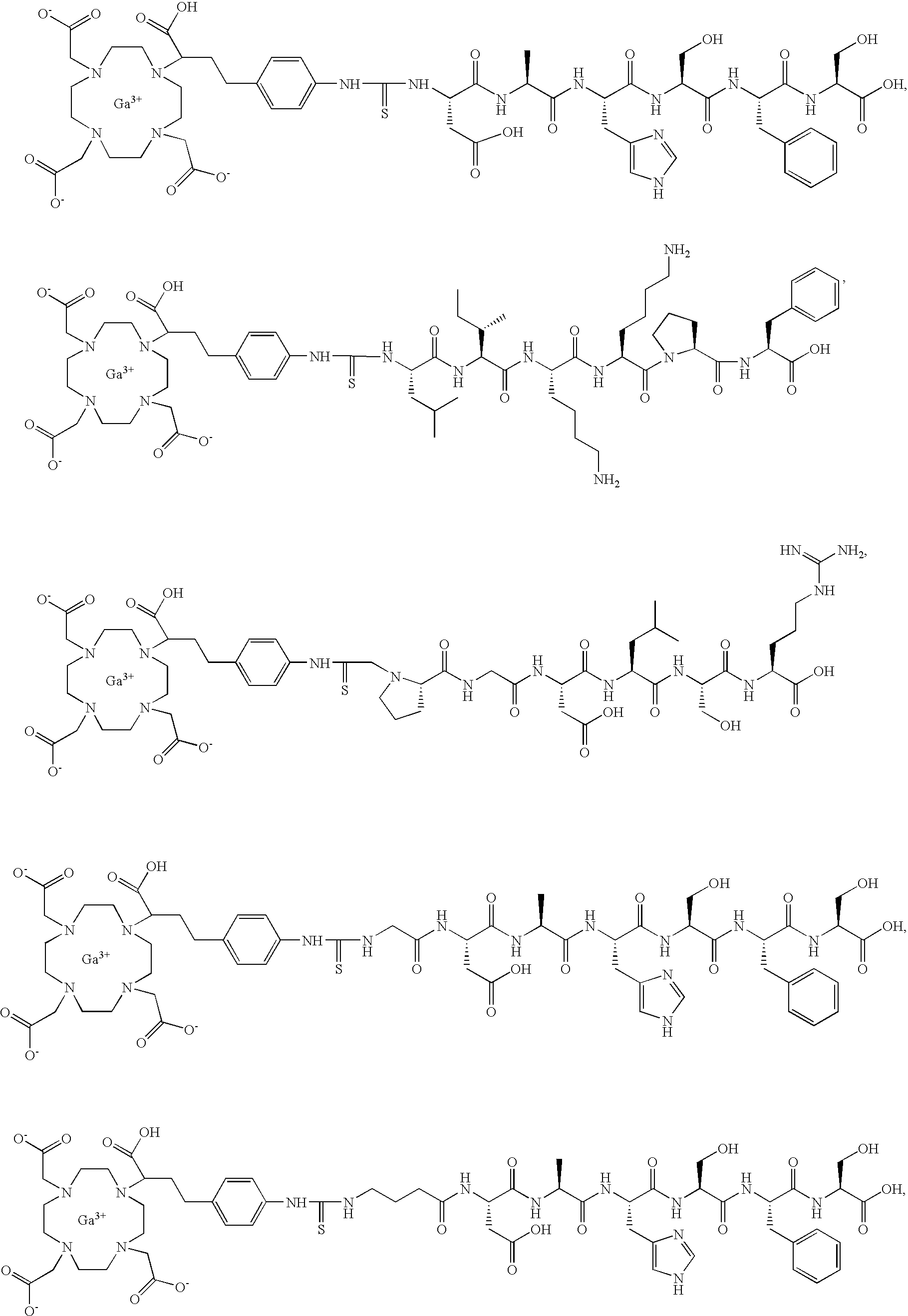

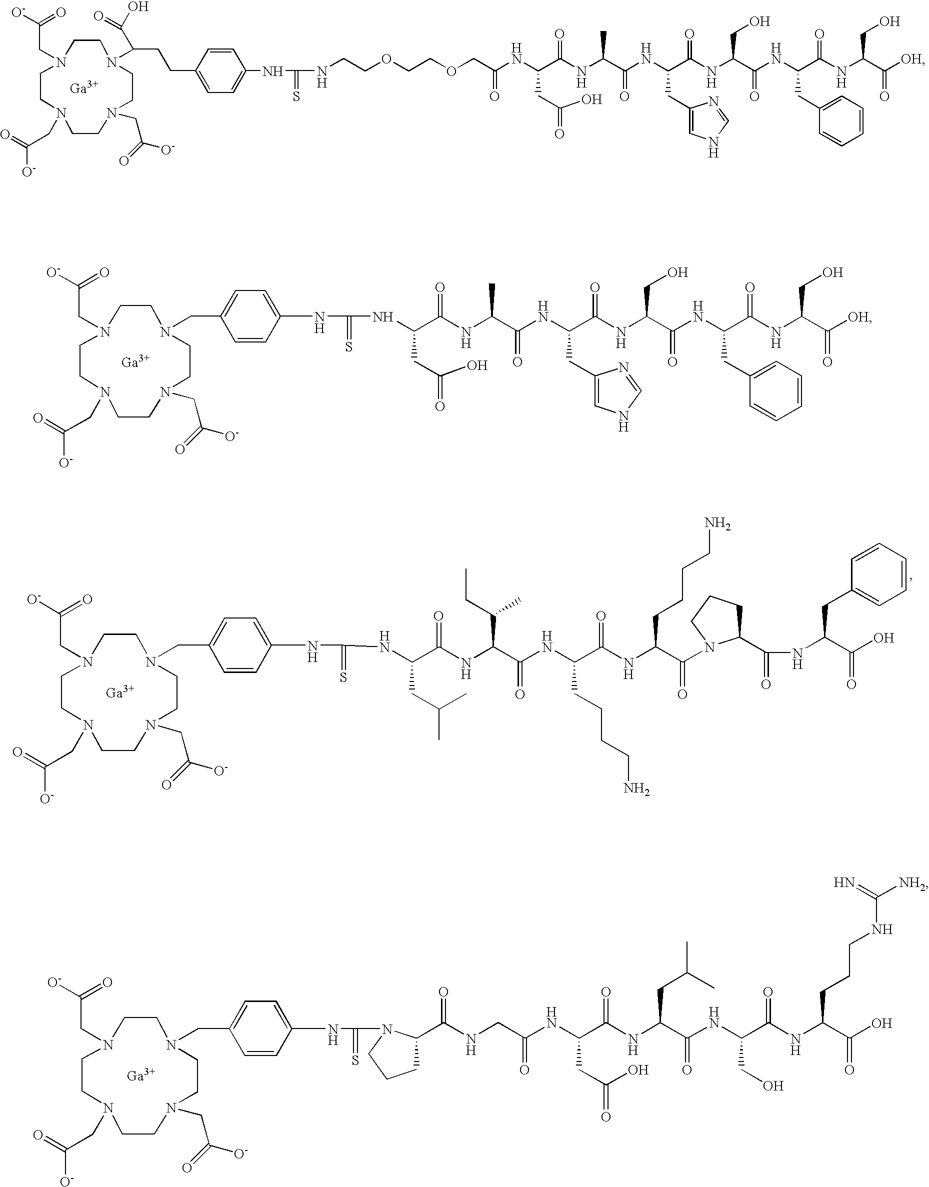

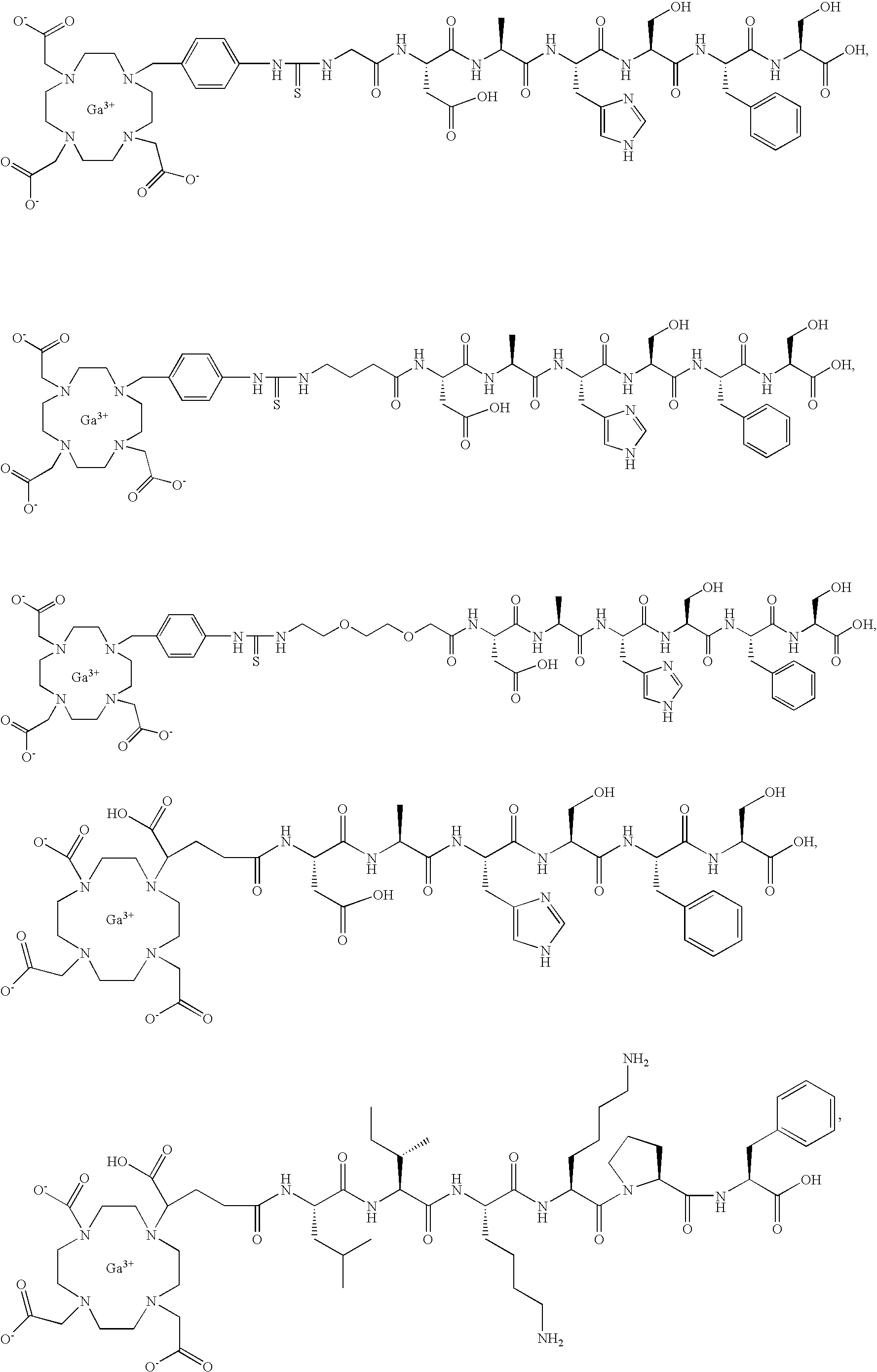

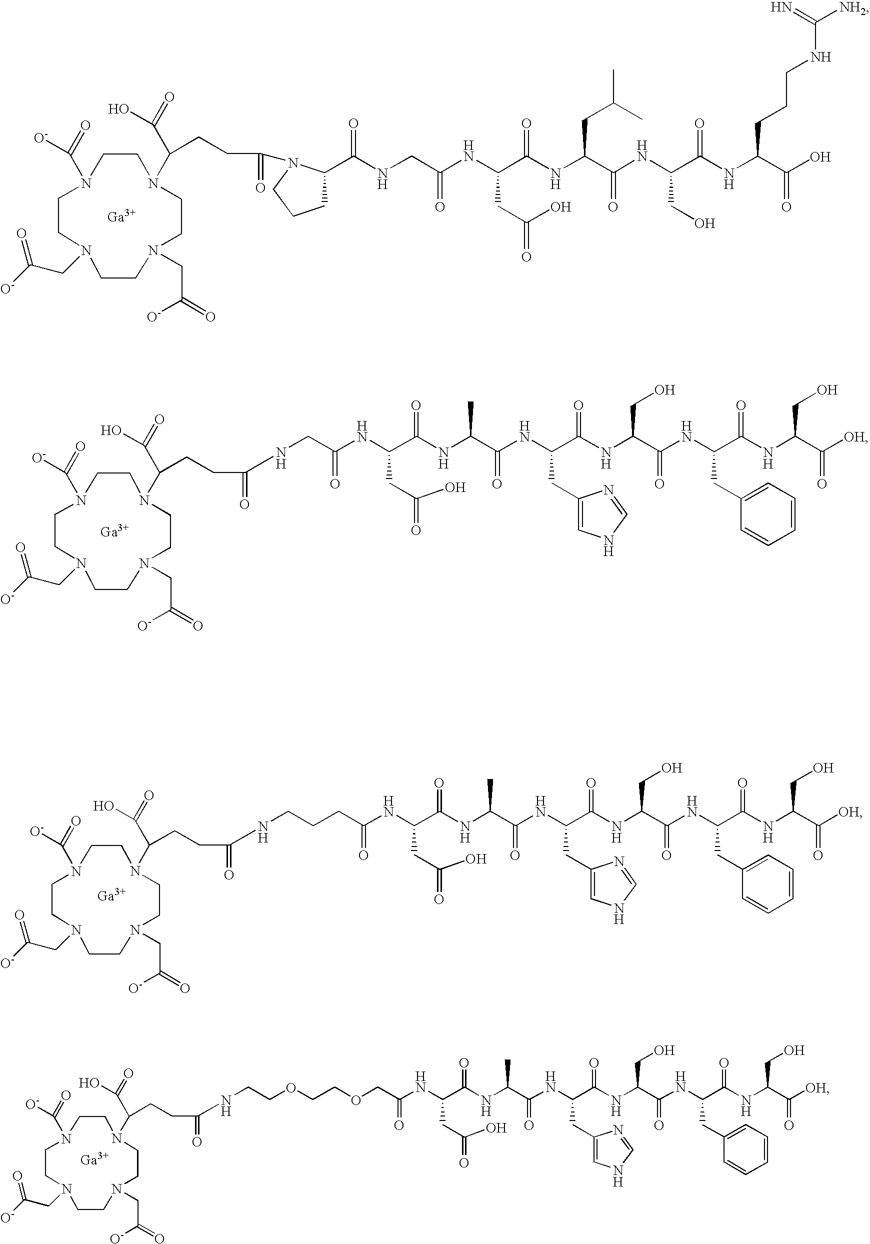

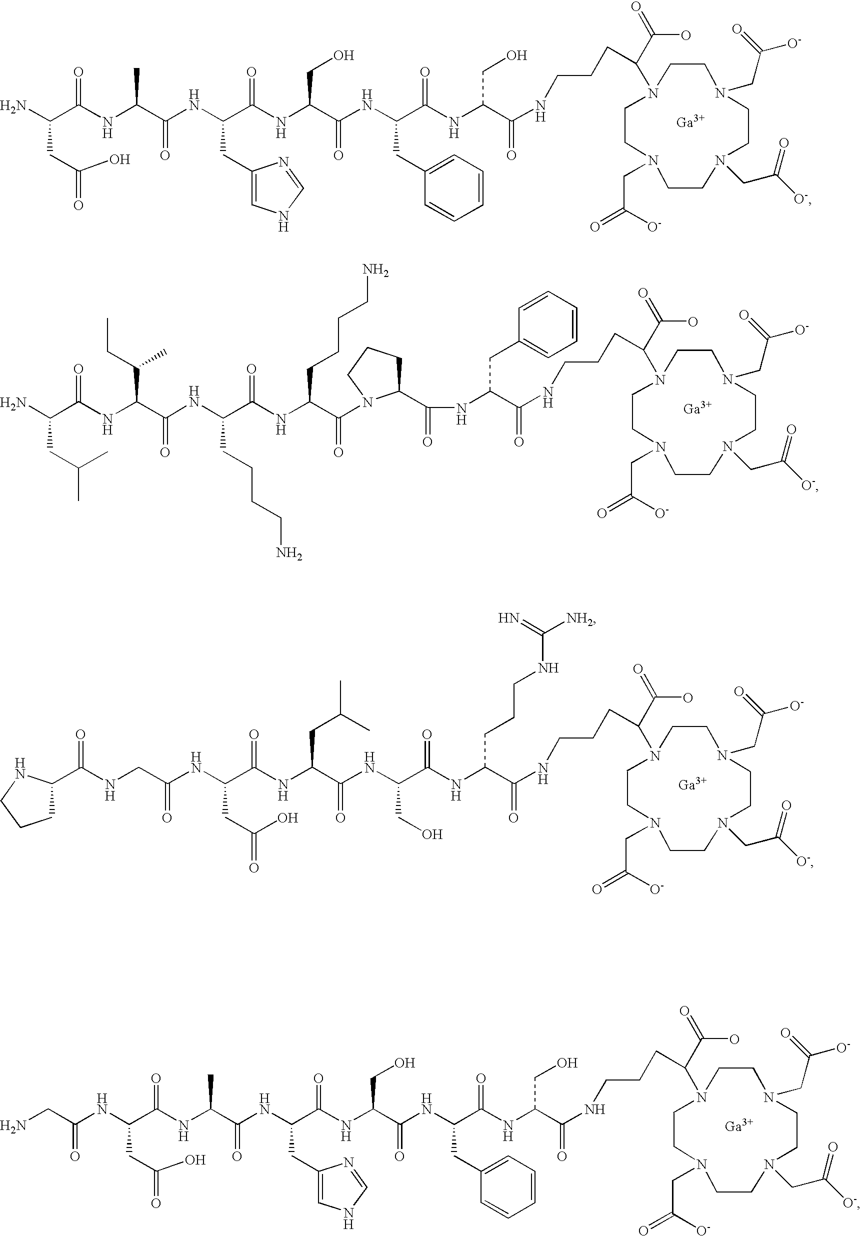

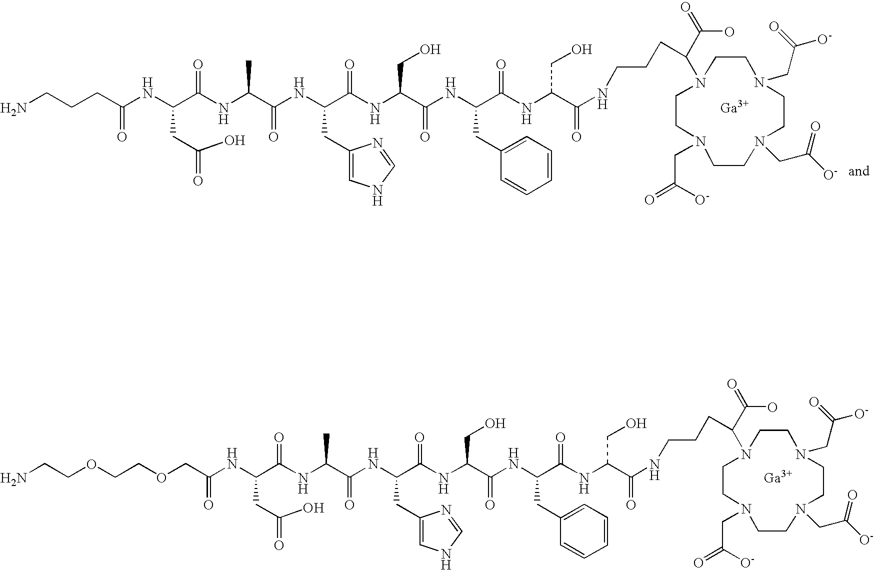

2. The compound as claimed in claim 1, in which Peptide is a peptide of formula LIKKPF (SEQ ID No 11).

3. The compound as claimed in claim 1, in which Signal is a chelate coupled to a paramagnetic metal M, M being chosen from Gd, Mn, Fe, Dy and Tm.

4. The compound as claimed in claim 1, in which Signal is a chelate coupled to the Ga68 radionucleide for PET imaging.

5. The compound as claimed in claim 1, in which Signal is a chelate which may or may not be coupled to a paramagnetic metal M or to a radionucleide, the chelate being chosen from: DOTA, DTPA, DO3A, HPDO3A, TRITA, TETA, BOPTA, NOTA, PCTA, DOTMA, AAZTA, HOPO and their derivatives.

6. The compound as claimed in claim 1, in which Linker represents:
a) a group of formula: Q1-1-Q2,
in which Q1 and Q2, which may be identical or different, represent O, S, NH, CO₂, —NHCO, CONH, NHCONH, NHCSNH, SO₂NH— or NHSO₂—,
and 1 represents an alkyl group, alkoxyalkyl group, polyalkoxyalkylene group, alkenyl group, alkynyl group, alkyl group interrupted with one or more squarates, with one or more aryls, or with one or more groups chosen from —NH—, —O—, —CO—, —NH(CO)—, —(CO)NH—, —O(CO)—, or —(OC)O—;
b) a (CH₂)ₙ, (CH₂)ₙ—CO—, —(CH₂)ₙNH—CO—, with n=2 to 10, (CH₂CH₂O)_{q}(CH₂)ᵣ—CO—, (CH₂CH₂O)_{q(CH}₂)ᵣ—NH—CO—, with q=1-10 and r=2-10, (CH₂)ₙ—CONH—, (CH₂)ₙ—CONH—PEG, (CH₂)ₙ—NH—, 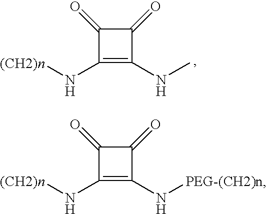
with
n=1 to 5, HOOC—CH₂—O—(CH₂)₂—O—(CH₂)₂—O—CH₂—COOH; HOOC—(CH₂)₂—CO₂—(CH₂)₂—OCO—(CH₂)₂—COOH; HOOC—CH(OH)—CH(OH)—COOH; HOOC—(CH₂)ₙ—COOH; NH₂—(CH₂)ₙ—NH₂, with n=1-20; NH₂—(CH₂)ₙ—CO₂H; or NH₂—CH₂—(CH₂—O—CH₂)ₙ—CO₂H, with n=1 to 10, group.

7. The compound as claimed in claim 1, in which Signal represents a superparamagnetic nanoparticle coated with a gem-bisphosphonate organic layer.

8. A composition for medical imaging, comprising at least one compound of general formula (I) as claimed in claim 1 and a pharmaceutically acceptable excipient.

9. A method for preparing the composition as claimed in claim 8, comprising the addition of a compound of formula (I) to an injectable medium comprising the pharmaceutically acceptable excipient.

10. Method for diagnosing diseases associated with apoptosis comprising the administration of an effective amount of a compound of general formula (I) as claimed in claim 1, to a patient in need thereof.

11. Method according to claim 10 wherein the diseases associated with apoptosis are chosen from chronic neurodegenerative diseases, ischemic diseases and inflammatory pathological conditions.

12. Method for diagnosing diseases associated with apoptosis comprising the administration of an apoptosis-targeting peptide of formula defined in claim 1 to a patient in need thereof.


SEQUENCE LISTING (11 sequences)

SEQ ID NO: 1 - protein, 6 aa, Artificial Sequence
  Chemically synthesized marker of apoptosis
LLKKPF

SEQ ID NO: 2 - protein, 6 aa, Artificial Sequence
  Chemically synthesized marker of apoptosis
DAHSFS

SEQ ID NO: 3 - protein, 6 aa, Artificial Sequence
  Chemically synthesized marker of apoptosis
PGDLST

SEQ ID NO: 4 - protein, 6 aa, Artificial Sequence
  Chemically synthesized marker of apoptosis
HGDLST

SEQ ID NO: 5 - protein, 6 aa, Artificial Sequence
  Chemically synthesized marker of apoptosis
VLGERG

SEQ ID NO: 6 - protein, 6 aa, Artificial Sequence
  Chemically synthesized marker of apoptosis
LIKKPF

SEQ ID NO: 7 - protein, 6 aa, Artificial Sequence
  Chemically synthesized marker of apoptosis
DAHSFS

SEQ ID NO: 8 - protein, 6 aa, Artificial Sequence
  Chemically synthesized marker of apoptosis
PGDLST

SEQ ID NO: 9 - protein, 6 aa, Artificial Sequence
  Chemically synthesized marker of apoptosis
HNDIST

SEQ ID NO: 10 - protein, 6 aa, Artificial Sequence
  Chemically synthesized marker of apoptosis
VLGDKG

SEQ ID NO: 11 - protein, 6 aa, Artificial Sequence
  Chemically synthesized marker of apoptosis
LIKKPF